(19) United States
(12) Patent Application Publication
(10) Pub. No.: US 2025/0111862 A1
(43) Pub. Date: Apr. 3, 2025

(54) DETECTING, PRESENTING, AND LOGGING RELEVANT HEALTH INFORMATION BASED ON A CONTEXT OF AN ELECTRONIC DEVICE IN A THREE-DIMENSIONAL ENVIRONMENT

(71) Applicant: Apple Inc., Cupertino, CA (US)

(72) Inventors: Ioana NEGOITA, Mountain View, CA (US); Brian W. TEMPLE, Santa Clara, CA (US); Ian PERRY, San Jose, CA (US); David LOEWENTHAL, Seattle, WA (US); Trent A. GREENE, Santa Clara, CA (US)

(21) Appl. No.: 18/803,279

(22) Filed: Aug. 13, 2024

Related U.S. Application Data
(60) Provisional application No. 63/586,341, filed on Sep. 28, 2023.

(51) Int. Cl. G10L 25/66; G06V 20/68; G06V 40/20

(52) U.S. Cl. CPC G10L 25/66; G06V 20/68; G06V 40/28

(57) ABSTRACT

In some examples, an electronic device presents, via a display, a representation of a prediction of a food being consumed by a user of the electronic device in a computer-generated environment. In some examples, the electronic device presents, via the display, an indication of possible non-compliance of medication in the computer-generated environment. In some examples, the electronic device initiates a smoking detection mode in response to the acquisition and processing of data from the user of the electronic device or from the physical environment of the user of the electronic device.

### CROSS-REFERENCE TO RELATED APPLICATIONS

This application claims the benefit of U.S. Provisional Application No. 63/586,341, filed Sep. 28, 2023, the content of which is herein incorporated by reference in its entirety for all purposes.

### FIELD OF THE DISCLOSURE

This relates generally to systems and methods of detecting, presenting, and logging relevant user health information based on a context of an electronic device in a three-dimensional environment.

### BACKGROUND OF THE DISCLOSURE

Some computer graphical environments provide two-dimensional and/or three-dimensional environments where at least some objects displayed for a user's viewing are virtual and generated by a computer. Some existing food logging applications require that a user manually enter a description of a food item and/or scan a barcode of the food item using their mobile device; however, these applications still do not provide an efficient way to automatically determine and log the particular food the user is consuming without requiring the user to provide further inputs to manually enter a description of a food item and/or scan a barcode of the food item using their mobile device. Additionally, these applications still do not provide a way to disambiguate between individual food items of a plurality of food items when determining which food item the user of the electronic device is consuming. Thus, there is a need for systems and methods that automatically detect and log the particular food item the user is consuming.

### SUMMARY OF THE DISCLOSURE

Some examples of the disclosure are directed to systems and methods for displaying a representation of a prediction of a food being consumed by a user of an electronic device in a computer-generated environment. In some examples, an electronic device is in communication with one or more displays and one or more input devices. In some examples, the electronic device detects that a user of the electronic device is initiating consumption of a first object. In some examples, in response to the electronic device detecting that the user of the electronic device is initiating consumption of the first object, the electronic device captures, using the one or more input devices, audio and one or more images of the first object and obtains a first prediction of the first object based on a sound print of the first object included in the audio. In some examples, in accordance with a determination that the first prediction of the first object satisfies one or more criteria, the electronic device initiates a process to analyze the one or more images of the first object.

Some examples of the disclosure are directed to systems and methods for displaying an indication of possible non-compliance of medication based on a context of an electronic device in a computer-generated environment. In some examples, an electronic device in communication with one or more displays and one or more input devices obtains medication information associated with a user of the electronic device. In some examples, while the medication information of the user indicates a dose within a predetermined period of time, the electronic device detects, via the one or more input devices, a change in contextual information. In some examples, in response to the electronic device detecting the change in contextual information, in accordance with a determination that one or more criteria are satisfied, including a criterion that is satisfied when the change in contextual information is associated with possible non-compliance of the medication, the electronic device presents an indication in a computer-generated environment of the possible non-compliance. In some examples, in response to the electronic device detecting the change in contextual information, in accordance with a determination that the one or more criteria are not satisfied, the electronic device foregoes presenting the indication in the computer-generated environment.

The full descriptions of these examples are provided in the Drawings and the Detailed Description, and it is understood that this Summary does not limit the scope of the disclosure in any way.

### BRIEF DESCRIPTION OF THE DRAWINGS

For improved understanding of the various examples described herein, reference should be made to the Detailed Description below along with the following drawings. Like reference numerals often refer to corresponding parts throughout the drawings.

FIG. 1 illustrates an electronic device presenting an extended reality environment according to some examples of the disclosure.

FIG. 2 illustrates a block diagram of an example architecture for a device according to some examples of the disclosure.

FIGS. 3A-3O illustrate examples of an electronic device displaying a representation of a prediction of a food being consumed by a user of the electronic device and an indication of possible non-compliance of medication in a computer-generated environment according to some examples of the disclosure.

FIGS. 4A-4C illustrate examples of an electronic device initiating a smoking detection mode according to some examples of the disclosure.

FIG. 5 is a flow diagram illustrating an example process for displaying a representation of a prediction of a food being consumed by a user of the electronic device in a computer-generated environment according to some examples of the disclosure.

FIG. 6 is a flow diagram illustrating an example process for displaying an indication of possible non-compliance of medication in a computer-generated environment according to some examples of the disclosure.

### DETAILED DESCRIPTION

Some examples of the disclosure are directed to systems and methods for displaying a representation of a prediction of a food being consumed by a user of an electronic device in a computer-generated environment. In some examples, an electronic device is in communication with one or more displays and one or more input devices. In some examples, the electronic device detects that a user of the electronic device is initiating consumption of a first object. In some examples, in response to the electronic device detecting that the user of the electronic device is initiating consumption of the first object, the electronic device captures, using the one or more input devices, audio and one or more images of the first object and obtains a first prediction of the first object based on a sound print of the first object included in the audio. In some examples, in accordance with a determination that the first prediction of the first object satisfies one or more criteria, the electronic device initiates a process to analyze the one or more images of the first object.

Some examples of the disclosure are directed to systems and methods for displaying an indication of possible non-compliance of medication in the computer-generated environment. In some examples, an electronic device in communication with one or more displays and one or more input devices obtains medication information associated with a user of the electronic device. In some examples, while the medication information of the user indicates a dose within a predetermined period of time, the electronic device detects, via the one or more input devices, a change in contextual information. In some examples, in response to the electronic device detecting the change in contextual information, in accordance with a determination that one or more criteria are satisfied, including a criterion that is satisfied when the change in contextual information is associated with possible non-compliance of the medication, the electronic device presents an indication in a computer-generated environment of the possible non-compliance. In some examples, in response to the electronic device detecting the change in contextual information, in accordance with a determination that the one or more criteria are not satisfied, the electronic device foregoes presenting the indication in the computer-generated environment.

In some examples, a three-dimensional object is displayed in a computer-generated three-dimensional environment with a particular orientation that controls one or more behaviors of the three-dimensional object (e.g., when the three-dimensional object is moved within the three-dimensional environment). In some examples, the orientation in which the three-dimensional object is displayed in the three-dimensional environment is selected by a user of the electronic device or automatically selected by the electronic device. For example, when initiating presentation of the three-dimensional object in the three-dimensional environment, the user may select a particular orientation for the three-dimensional object or the electronic device may automatically select the orientation for the three-dimensional object (e.g., based on a type of the three-dimensional object).

In some examples, a three-dimensional object can be displayed in the three-dimensional environment in a world-locked orientation, a body-locked orientation, a tilt-locked orientation, or a head-locked orientation, as described below. As used herein, an object that is displayed in a body-locked orientation in a three-dimensional environment has a distance and orientation offset relative to a portion of the user's body (e.g., the user's torso). Alternatively, in some examples, a body-locked object has a fixed distance from the user without the orientation of the content being referenced to any portion of the user's body (e.g., may be displayed in the same cardinal direction relative to the user, regardless of head and/or body movement). Additionally or alternatively, in some examples, the body-locked object may be configured to always remain gravity or horizon (e.g., normal to gravity) aligned, such that head and/or body changes in the roll direction would not cause the body-locked object to move within the three-dimensional environment. Rather, translational movement in either configuration would cause the body-locked object to be repositioned within the three-dimensional environment to maintain the distance offset.

As used herein, an object that is displayed in a head-locked orientation in a three-dimensional environment has a distance and orientation offset relative to the user's head. In some examples, a head-locked object moves within the three-dimensional environment as the user's head moves (as the viewpoint of the user changes).

As used herein, an object that is displayed in a world-locked orientation in a three-dimensional environment does not have a distance or orientation offset relative to the user.

As used herein, an object that is displayed in a tilt-locked orientation in a three-dimensional environment (referred to herein as a tilt-locked object) has a distance offset relative to the user, such as a portion of the user's body (e.g., the user's torso) or the user's head. In some examples, a tilt-locked object is displayed at a fixed orientation relative to the three-dimensional environment. In some examples, a tilt-locked object moves according to a polar (e.g., spherical) coordinate system centered at a pole through the user (e.g., the user's head). For example, the tilt-locked object is moved in the three-dimensional environment based on movement of the user's head within a spherical space surrounding (e.g., centered at) the user's head. Accordingly, if the user tilts their head (e.g., upward or downward in the pitch direction) relative to gravity, the tilt-locked object would follow the head tilt and move radially along a sphere, such that the tilt-locked object is repositioned within the three-dimensional environment to be the same distance offset relative to the user as before the head tilt while optionally maintaining the same orientation relative to the three-dimensional environment. In some examples, if the user moves their head in the roll direction (e.g., clockwise or counterclockwise) relative to gravity, the tilt-locked object is not repositioned within the three-dimensional environment.

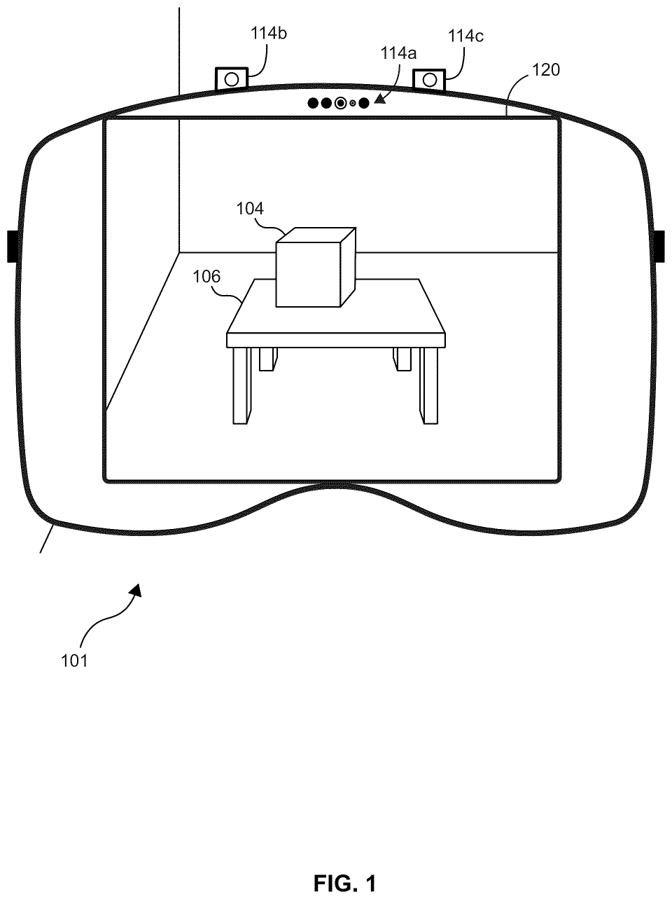

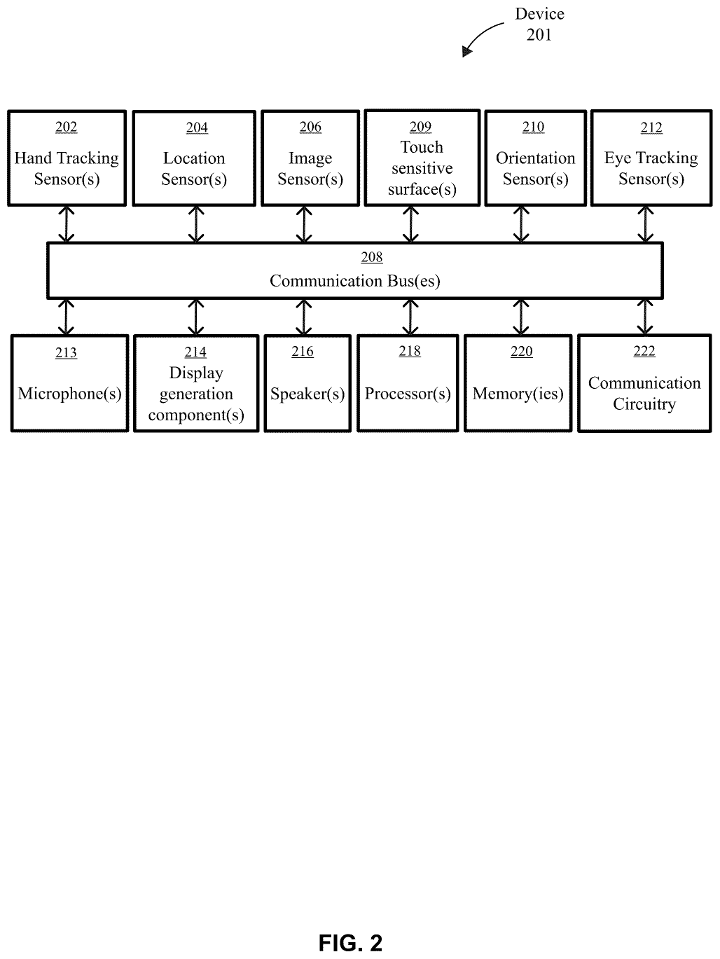

FIG. 1 illustrates an electronic device 101 presenting an extended reality (XR) environment (e.g., a computer-generated environment optionally including representations of physical and/or virtual objects) according to some examples of the disclosure. In some examples, as shown in FIG. 1, electronic device 101 is a head-mounted display or other head-mountable device configured to be worn on a head of a user of the electronic device 101. Examples of electronic device 101 are described below with reference to the architecture block diagram of FIG. 2. As shown in FIG. 1, electronic device 101 and table 106 are located in a physical environment. The physical environment may include physical features such as a physical surface (e.g., floor, walls) or a physical object (e.g., table, lamp, etc.). In some examples, electronic device 101 may be configured to detect and/or capture images of physical environment including table 106 (illustrated in the field of view of electronic device 101).

In some examples, as shown in FIG. 1, electronic device 101 includes one or more internal image sensors 114a oriented towards a face of the user (e.g., eye tracking cameras described below with reference to FIG. 2). In some examples, internal image sensors 114a are used for eye tracking (e.g., detecting a gaze of the user). Internal image sensors 114a are optionally arranged on the left and right portions of display 120 to enable eye tracking of the user's left and right eyes. In some examples, electronic device 101 also includes external image sensors 114b and 114c facing outwards from the user to detect and/or capture the physical environment of the electronic device 101 and/or movements of the user's hands or other body parts.

In some examples, display 120 has a field of view visible to the user (e.g., that may or may not correspond to a field of view of external image sensors 114b and 114c). Because display 120 is optionally part of a head-mounted device, the field of view of display 120 is optionally the same as or similar to the field of view of the user's eyes. In other examples, the field of view of display 120 may be smaller than the field of view of the user's eyes. In some examples, electronic device 101 may be an optical see-through device in which display 120 is a transparent or translucent display through which portions of the physical environment may be directly viewed. In some examples, display 120 may be included within a transparent lens and may overlap all or only a portion of the transparent lens. In other examples, electronic device may be a video-passthrough device in which display 120 is an opaque display configured to display images of the physical environment captured by external image sensors 114b and 114c.

In some examples, in response to a trigger, the electronic device 101 may be configured to display a virtual object 104 in the XR environment represented by a cube illustrated in FIG. 1, which is not present in the physical environment, but is displayed in the XR environment positioned on the top of real-world table 106 (or a representation thereof). Optionally, virtual object 104 can be displayed on the surface of the table 106 in the XR environment displayed via the display 120 of the electronic device 101 in response to detecting the planar surface of table 106 in the physical environment 100.

It should be understood that virtual object 104 is a representative virtual object and one or more different virtual objects (e.g., of various dimensionality such as two-dimensional or other three-dimensional virtual objects) can be included and rendered in a three-dimensional XR environment. For example, the virtual object can represent an application or a user interface displayed in the XR environment. In some examples, the virtual object can represent content corresponding to the application and/or displayed via the user interface in the XR environment. In some examples, the virtual object 104 is optionally configured to be interactive and responsive to user input (e.g., air gestures, such as air pinch gestures, air tap gestures, and/or air touch gestures), such that a user may virtually touch, tap, move, rotate, or otherwise interact with, the virtual object 104.

In some examples, displaying an object in a three-dimensional environment may include interaction with one or more user interface objects in the three-dimensional environment. For example, initiation of display of the object in the three-dimensional environment can include interaction with one or more virtual options/affordances displayed in the three-dimensional environment. In some examples, a user's gaze may be tracked by the electronic device as an input for identifying one or more virtual options/affordances targeted for selection when initiating display of an object in the three-dimensional environment. For example, gaze can be used to identify one or more virtual options/affordances targeted for selection using another selection input. In some examples, a virtual option/affordance may be selected using hand-tracking input detected via an input device in communication with the electronic device. In some examples, objects displayed in the three-dimensional environment may be moved and/or reoriented in the three-dimensional environment in accordance with movement input detected via the input device.

In the discussion that follows, an electronic device that is in communication with a display generation component and one or more input devices is described. It should be understood that the electronic device optionally is in communication with one or more other physical user-interface devices, such as a touch-sensitive surface, a physical keyboard, a mouse, a joystick, a hand tracking device, an eye tracking device, a stylus, etc. Further, as described above, it should be understood that the described electronic device, display and touch-sensitive surface are optionally distributed amongst two or more devices. Therefore, as used in this disclosure, information displayed on the electronic device or by the electronic device is optionally used to describe information outputted by the electronic device for display on a separate display device (touch-sensitive or not). Similarly, as used in this disclosure, input received on the electronic device (e.g., touch input received on a touch-sensitive surface of the electronic device, or touch input received on the surface of a stylus) is optionally used to describe input received on a separate input device, from which the electronic device receives input information.

The device typically supports a variety of applications, such as one or more of the following: a drawing application, a presentation application, a word processing application, a website creation application, a disk authoring application, a spreadsheet application, a gaming application, a telephone application, a video conferencing application, an e-mail application, an instant messaging application, a workout support application, a photo management application, a digital camera application, a digital video camera application, a web browsing application, a digital music player application, a television channel browsing application, and/or a digital video player application.

FIG. 2 illustrates a block diagram of an example architecture for a device 201 according to some examples of the disclosure. In some examples, device 201 includes one or more electronic devices. For example, the electronic device 201 may be a portable device, an auxiliary device in communication with another device, a head-mounted display, etc., respectively. In some examples, electronic device 201 corresponds to electronic device 101 described above with reference to FIG. 1.

As illustrated in FIG. 2, the electronic device 201 optionally includes various sensors, such as one or more hand tracking sensors 202, one or more location sensors 204, one or more image sensors 206 (optionally corresponding to internal image sensors 114a and/or external image sensors 114b and 114c in FIG. 1), one or more touch-sensitive surfaces 209, one or more motion and/or orientation sensors 210, one or more eye tracking sensors 212, one or more microphones 213 or other audio sensors, one or more body tracking sensors (e.g., torso and/or head tracking sensors), one or more displays 214, optionally corresponding to display 120 in FIG. 1, one or more speakers 216, one or more processors 218, one or more memories 220, and/or communication circuitry 222. One or more communication buses 208 are optionally used for communication between the above-mentioned components of electronic devices 201.

Communication circuitry 222 optionally includes circuitry for communicating with electronic devices, networks, such as the Internet, intranets, a wired network and/or a wireless network, cellular networks, and wireless local area networks (LANs). Communication circuitry 222 optionally includes circuitry for communicating using near-field communication (NFC) and/or short-range communication, such as Bluetooth®.

Processor(s) 218 include one or more general processors, one or more graphics processors, and/or one or more digital signal processors. In some examples, memory 220 is a non-transitory computer-readable storage medium (e.g., flash memory, random access memory, or other volatile or non-volatile memory or storage) that stores computer-readable instructions configured to be executed by processor(s) 218 to perform the techniques, processes, and/or methods described below. In some examples, memory 220 can include more than one non-transitory computer-readable storage medium. A non-transitory computer-readable storage medium can be any medium (e.g., excluding a signal) that can tangibly contain or store computer-executable instructions for use by or in connection with the instruction execution system, apparatus, or device. In some examples, the storage medium is a transitory computer-readable storage medium. In some examples, the storage medium is a non-transitory computer-readable storage medium. The non-transitory computer-readable storage medium can include, but is not limited to, magnetic, optical, and/or semiconductor storages. Examples of such storage include magnetic disks, optical discs based on compact disc (CD), digital versatile disc (DVD), or Blu-ray technologies, as well as persistent solid-state memory such as flash, solid-state drives, and the like.

In some examples, display generation component(s) 214 include a single display (e.g., a liquid-crystal display (LCD), organic light-emitting diode (OLED), or other types of display). In some examples, display generation component(s) 214 includes multiple displays. In some examples, display generation component(s) 214 can include a display with touch capability (e.g., a touch screen), a projector, a holographic projector, a retinal projector, a transparent or translucent display, etc. In some examples, electronic device 201 includes touch-sensitive surface(s) 209, respectively, for receiving user inputs, such as tap inputs and swipe inputs or other gestures. In some examples, display generation component(s) 214 and touch-sensitive surface(s) 209 form touch-sensitive display(s) (e.g., a touch screen integrated with electronic device 201 or external to electronic device 201 that is in communication with electronic device 201).

Electronic device 201 optionally includes image sensor(s) 206. Image sensors(s) 206 optionally include one or more visible light image sensors, such as charged coupled device (CCD) sensors, and/or complementary metal-oxide-semiconductor (CMOS) sensors operable to obtain images of physical objects from the real-world environment. Image sensor(s) 206 also optionally include one or more infrared (IR) sensors, such as a passive or an active IR sensor, for detecting infrared light from the real-world environment. For example, an active IR sensor includes an IR emitter for emitting infrared light into the real-world environment. Image sensor(s) 206 also optionally include one or more cameras configured to capture movement of physical objects in the real-world environment. Image sensor(s) 206 also optionally include one or more depth sensors configured to detect the distance of physical objects from electronic device 201. In some examples, information from one or more depth sensors can allow the device to identify and differentiate objects in the real-world environment from other objects in the real-world environment. In some examples, one or more depth sensors can allow the device to determine the texture and/or topography of objects in the real-world environment.

In some examples, electronic device 201 uses CCD sensors, event cameras, and depth sensors in combination to detect the physical environment around electronic device 201. In some examples, image sensor(s) 206 include a first image sensor and a second image sensor. The first image sensor and the second image sensor work in tandem and are optionally configured to capture different information of physical objects in the real-world environment. In some examples, the first image sensor is a visible light image sensor and the second image sensor is a depth sensor. In some examples, electronic device 201 uses image sensor(s) 206 to detect the position and orientation of electronic device 201 and/or display generation component(s) 214 in the real-world environment. For example, electronic device 201 uses image sensor(s) 206 to track the position and orientation of display generation component(s) 214 relative to one or more fixed objects in the real-world environment.

In some examples, electronic device 201 includes microphone(s) 213 or other audio sensors. Electronic device 201 optionally uses microphone(s) 213 to detect sound from the user and/or the real-world environment of the user. In some examples, microphone(s) 213 includes an array of microphones (a plurality of microphones) that optionally operate in tandem, such as to identify ambient noise or to locate the source of sound in space of the real-world environment.

Electronic device 201 includes location sensor(s) 204 for detecting a location of electronic device 201 and/or display generation component(s) 214. For example, location sensor(s) 204 can include a GPS receiver that receives data from one or more satellites and allows electronic device 201 to determine the device's absolute position in the physical world.

Electronic device 201 includes orientation sensor(s) 210 for detecting orientation and/or movement of electronic device 201 and/or display generation component(s) 214. For example, electronic device 201 uses orientation sensor(s) 210 to track changes in the position and/or orientation of electronic device 201 and/or display generation component(s) 214, such as with respect to physical objects in the real-world environment. Orientation sensor(s) 210 optionally include one or more gyroscopes and/or one or more accelerometers.

Electronic device 201 includes hand tracking sensor(s) 202 and/or eye tracking sensor(s) 212 (and/or other body tracking sensor(s), such as leg, torso and/or head tracking sensor(s)), in some examples. Hand tracking sensor(s) 202 are configured to track the position/location of one or more portions of the user's hands, and/or motions of one or more portions of the user's hands with respect to the extended reality environment, relative to the display generation component(s) 214, and/or relative to another defined coordinate system. Eye tracking sensor(s) 212 are configured to track the position and movement of a user's gaze (eyes, face, or head, more generally) with respect to the real-world or extended reality environment and/or relative to the display generation component(s) 214. In some examples, hand tracking sensor(s) 202 and/or eye tracking sensor(s) 212 are implemented together with the display generation component(s) 214. In some examples, the hand tracking sensor(s) 202 and/or eye tracking sensor(s) 212 are implemented separate from the display generation component(s) 214.

In some examples, the hand tracking sensor(s) 202 (and/or other body tracking sensor(s), such as leg, torso and/or head tracking sensor(s)) can use image sensor(s) 206 (e.g., one or more IR cameras, 3D cameras, depth cameras, etc.) that capture three-dimensional information from the real-world including one or more body parts (e.g., leg, torso, head, or hand of a human user). In some examples, the hands can be resolved with sufficient resolution to distinguish fingers and their respective positions. In some examples, one or more image sensors 206 are positioned relative to the user to define a field of view of the image sensor(s) 206 and an interaction space in which finger/hand position, orientation and/or movement captured by the image sensors are used as inputs (e.g., to distinguish from a user's resting hand or other hands of other persons in the real-world environment). Tracking the fingers/hands for input (e.g., gestures, touch, tap, etc.) can be advantageous in that it does not require the user to touch, hold or wear any sort of beacon, sensor, or other marker.

In some examples, eye tracking sensor(s) 212 includes at least one eye tracking camera (e.g., infrared (IR) cameras) and/or illumination sources (e.g., IR light sources, such as LEDs) that emit light towards a user's eyes. The eye tracking cameras may be pointed towards a user's eyes to receive reflected IR light from the light sources directly or indirectly from the eyes. In some examples, both eyes are tracked separately by respective eye tracking cameras and illumination sources, and a focus/gaze can be determined from tracking both eyes. In some examples, one eye (e.g., a dominant eye) is tracked by one or more respective eye tracking cameras/illumination sources.

Electronic device 201 is not limited to the components and configuration of FIG. 2, but can include fewer, other, or additional components in multiple configurations. In some examples, electronic device 201 can be implemented between two electronic devices (e.g., as a system). In some such examples, each of (or more) electronic device may each include one or more of the same components discussed above, such as various sensors, one or more displays, one or more speakers, one or more processors, one or more memories, and/or communication circuitry. A person or persons using electronic device 201, is optionally referred to herein as a user or users of the device.

Attention is now directed towards examples of displaying of a representation of a prediction of a food being consumed by a user of the electronic device and an indication of possible non-compliance of medication based on a context of an electronic device in a computer-generated environment.

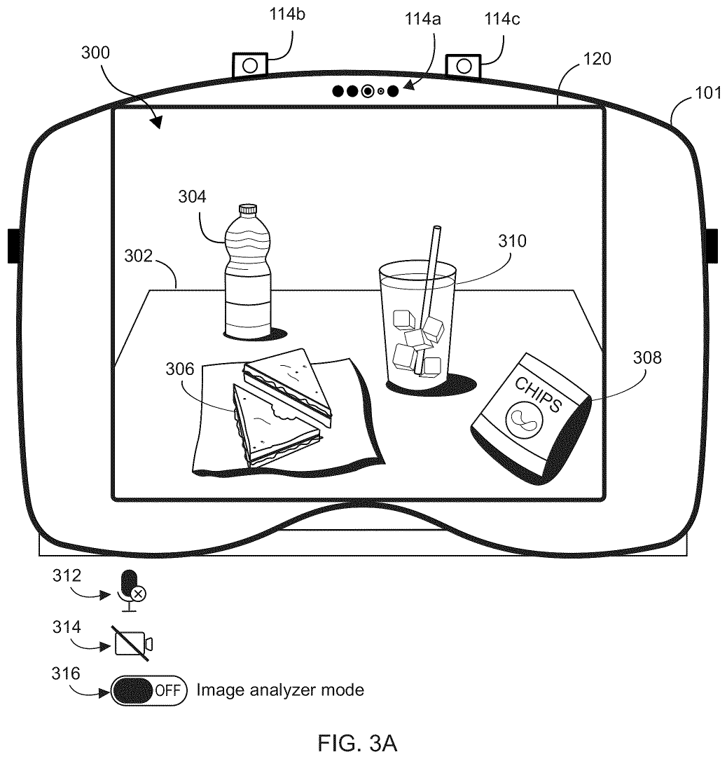

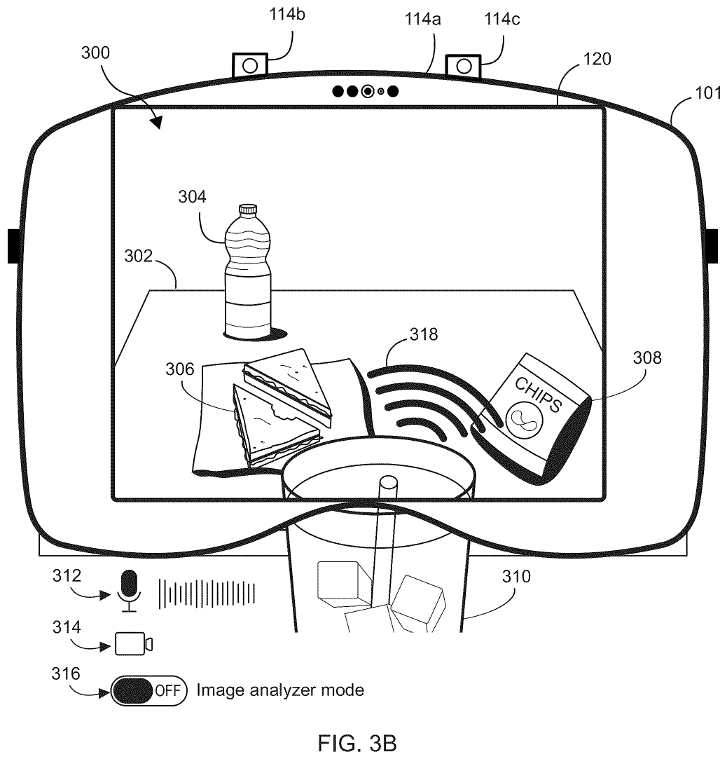

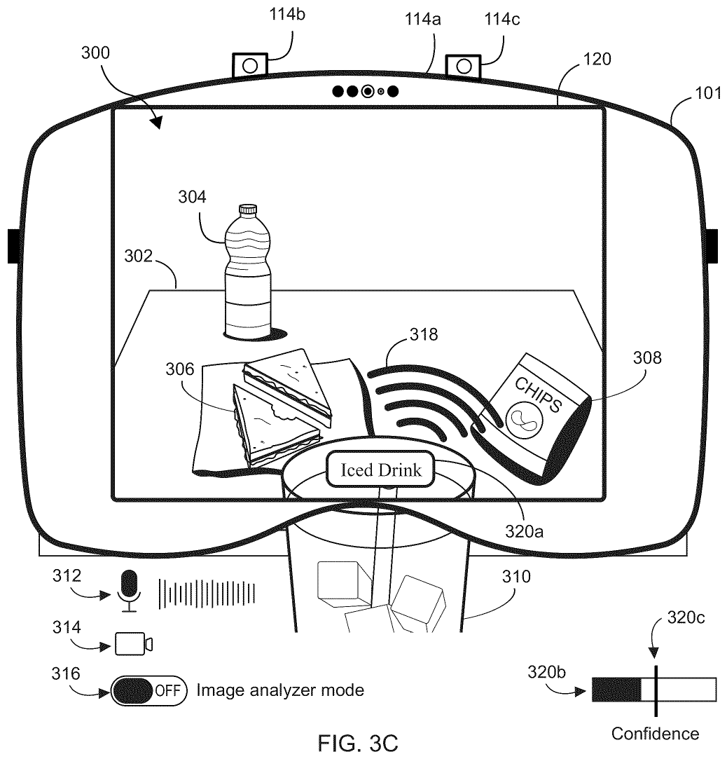

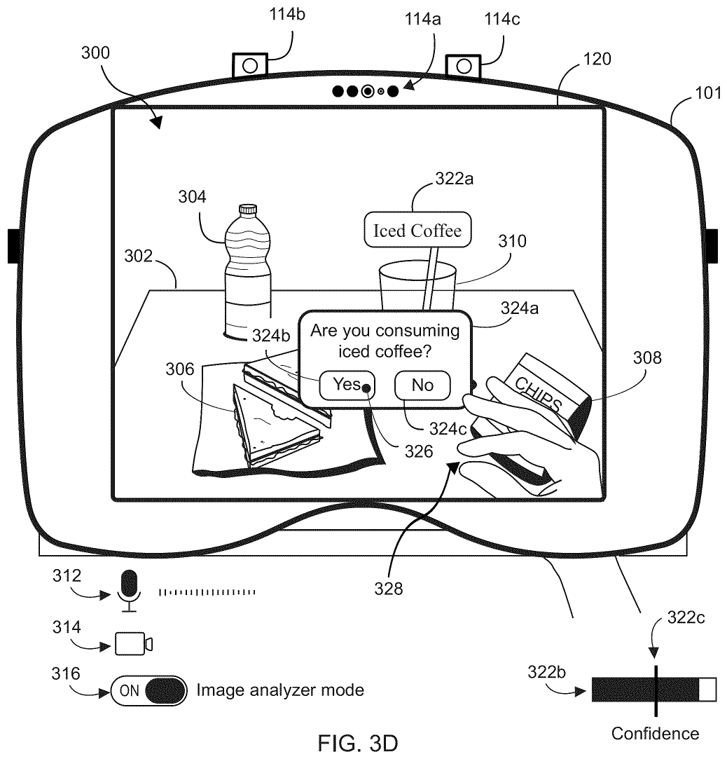

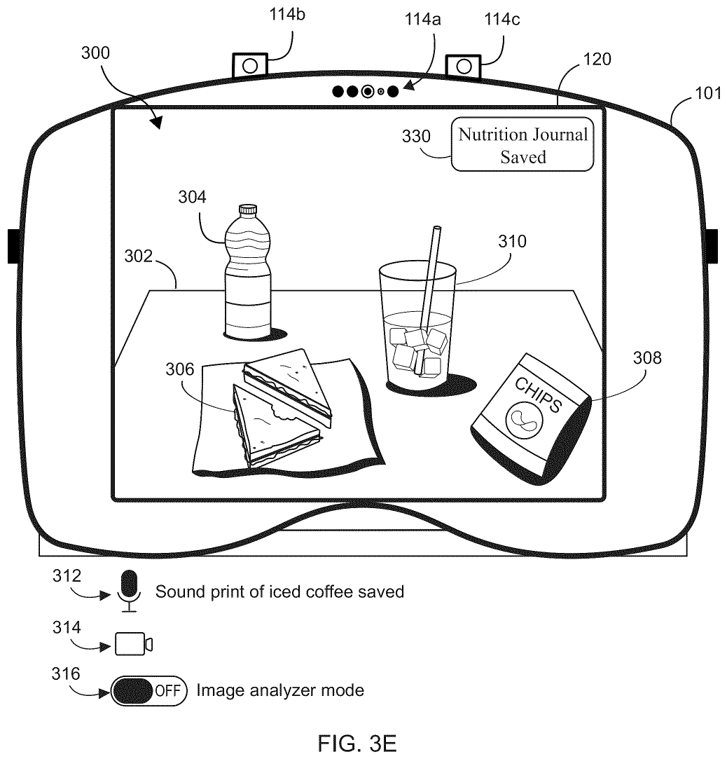

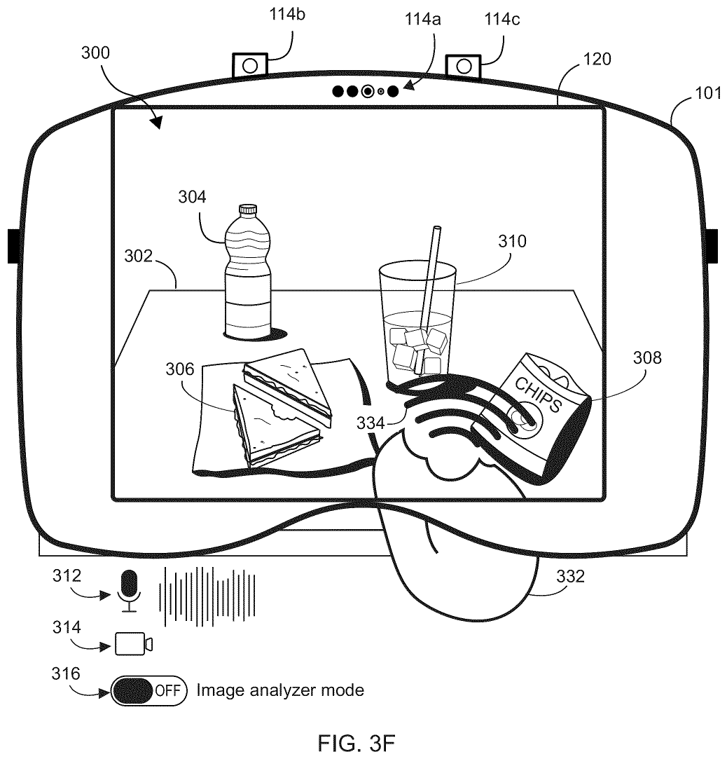

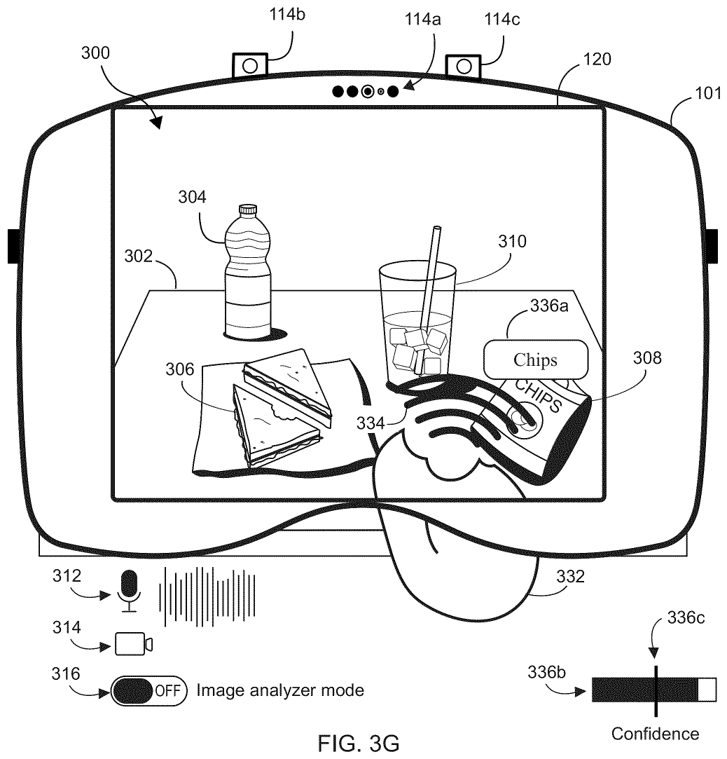

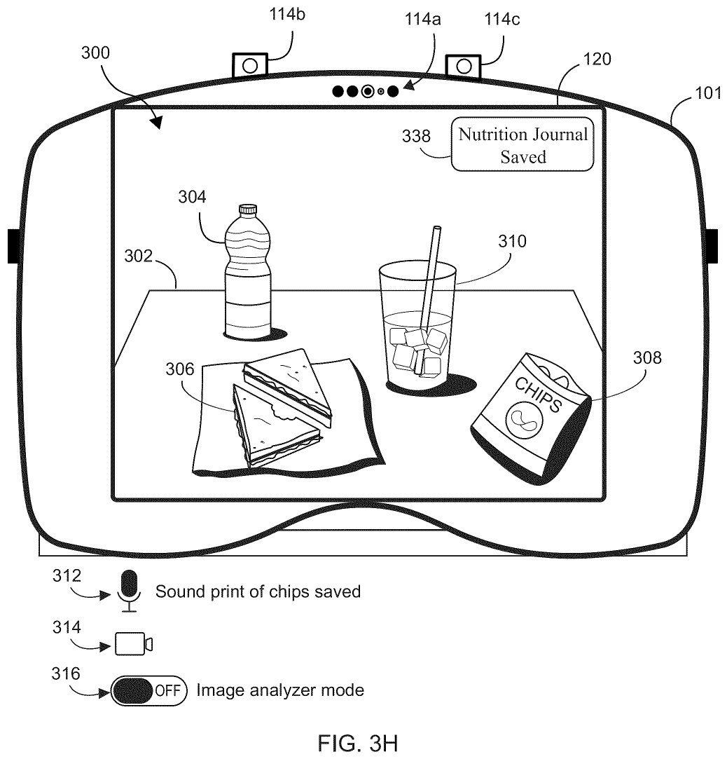

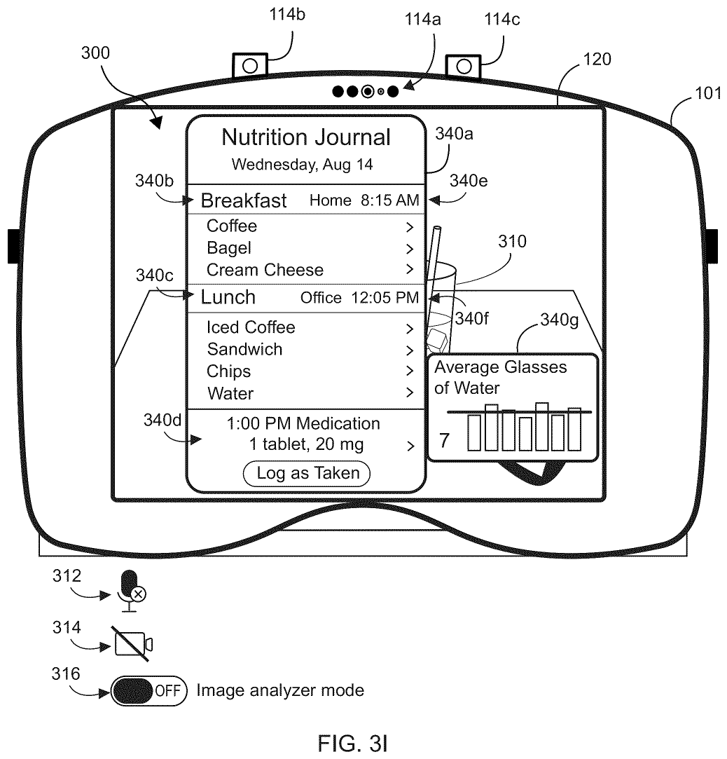

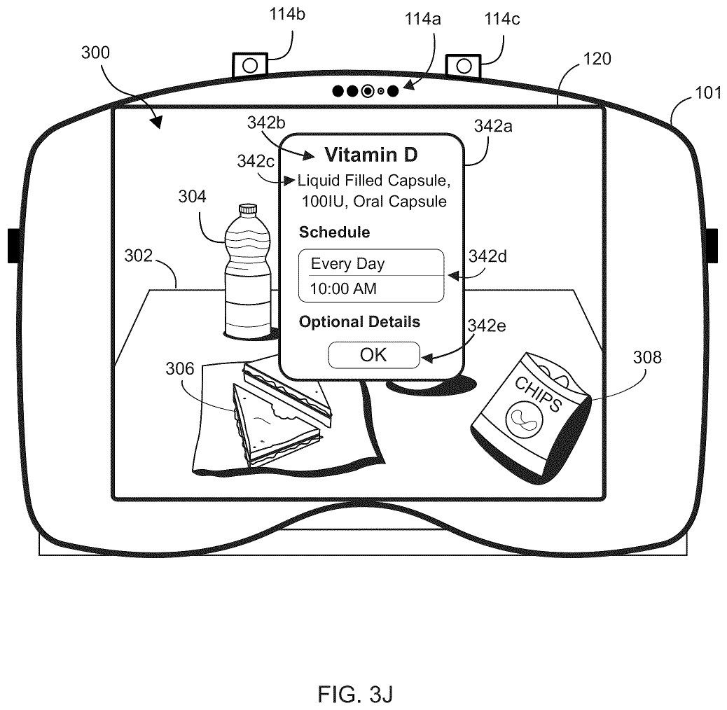

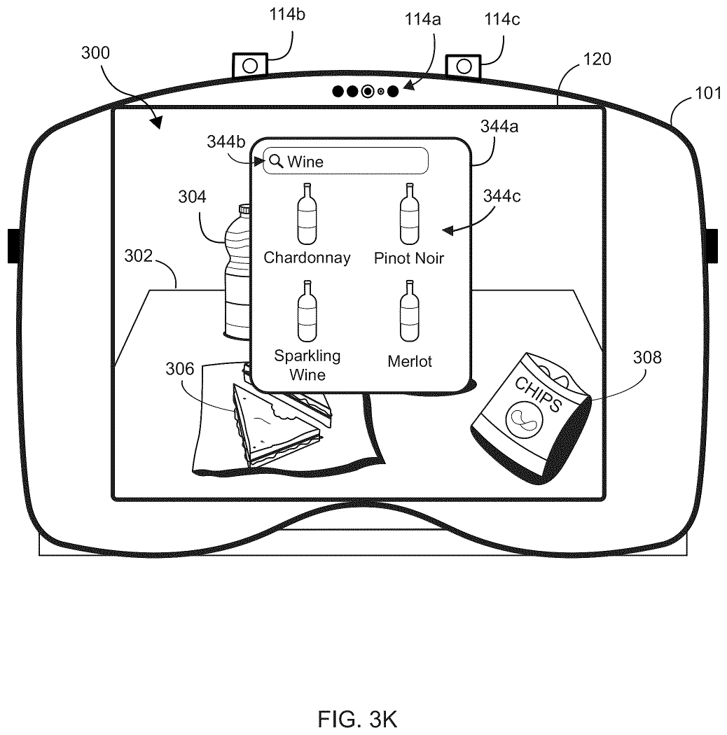

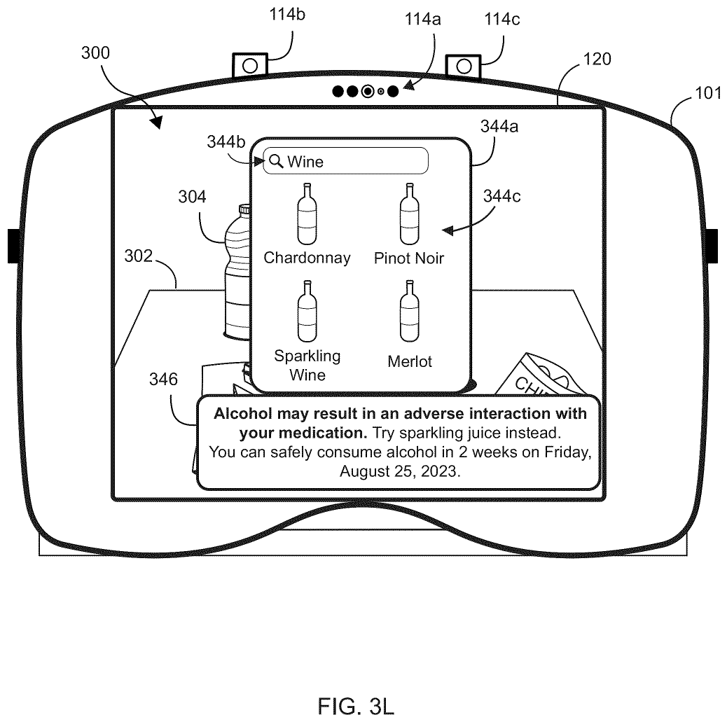

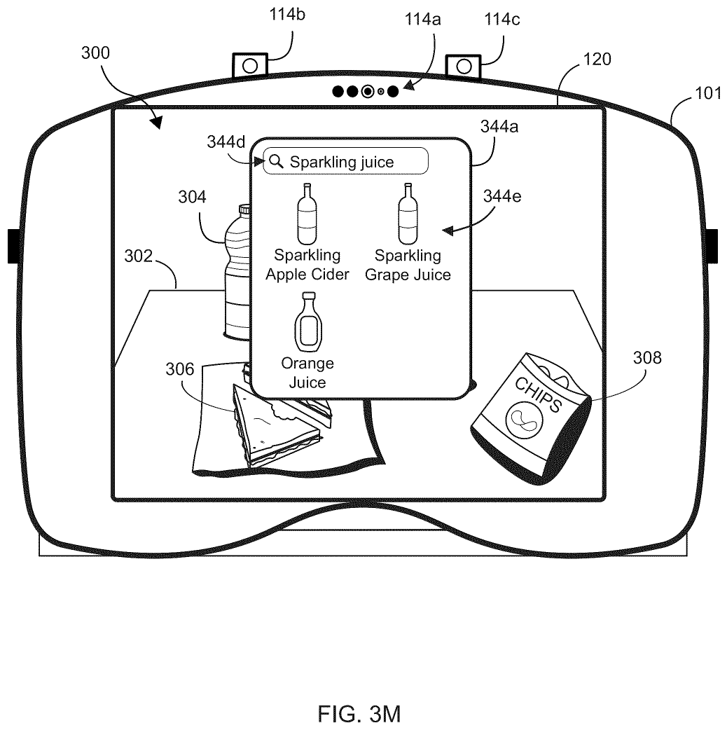

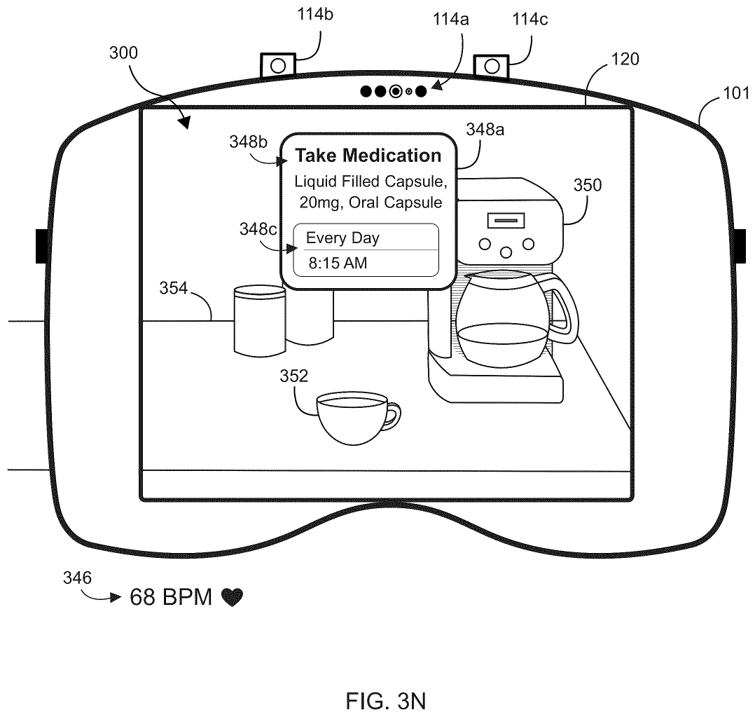

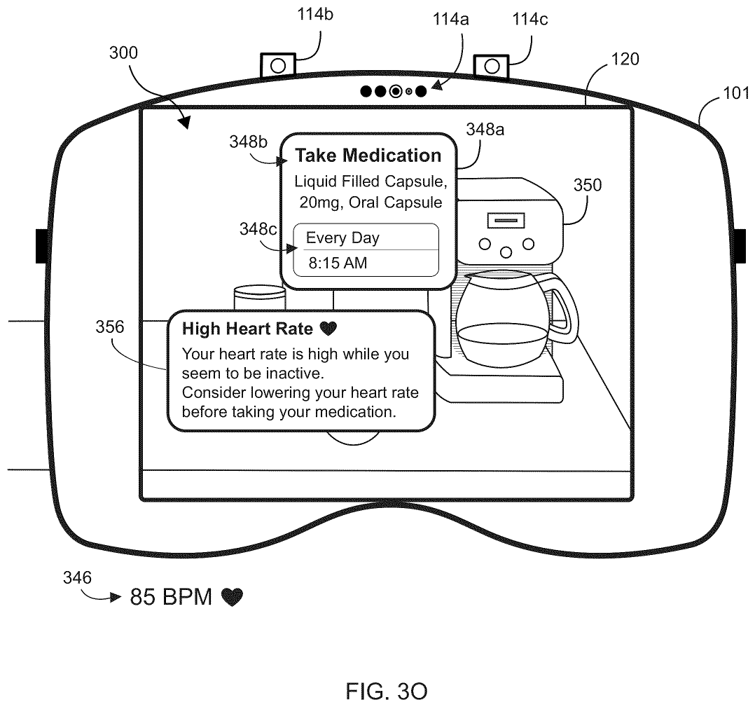

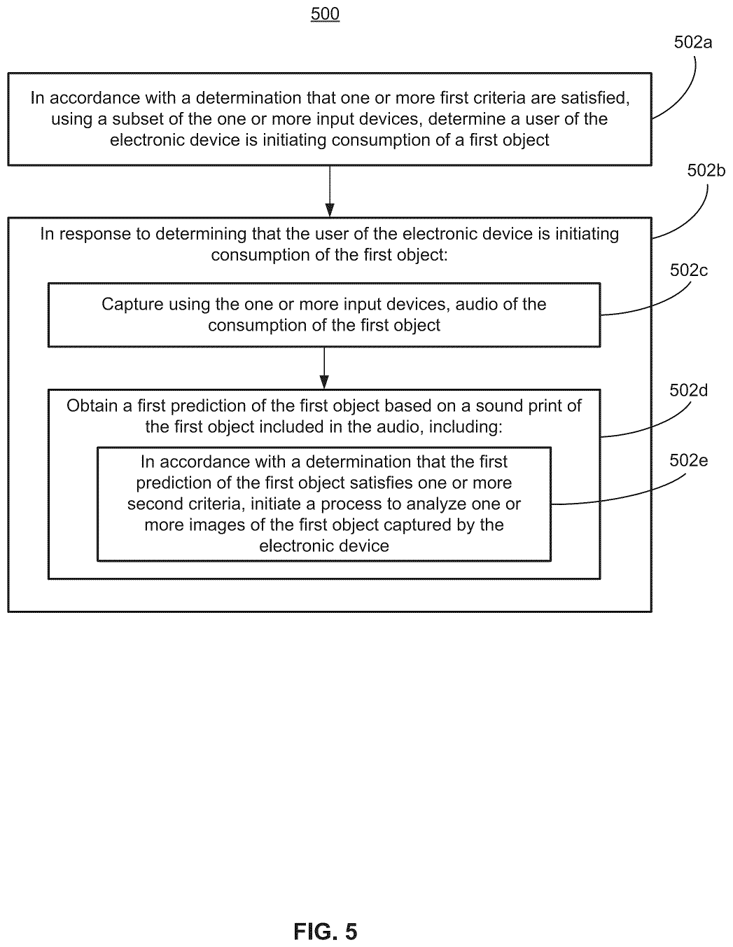

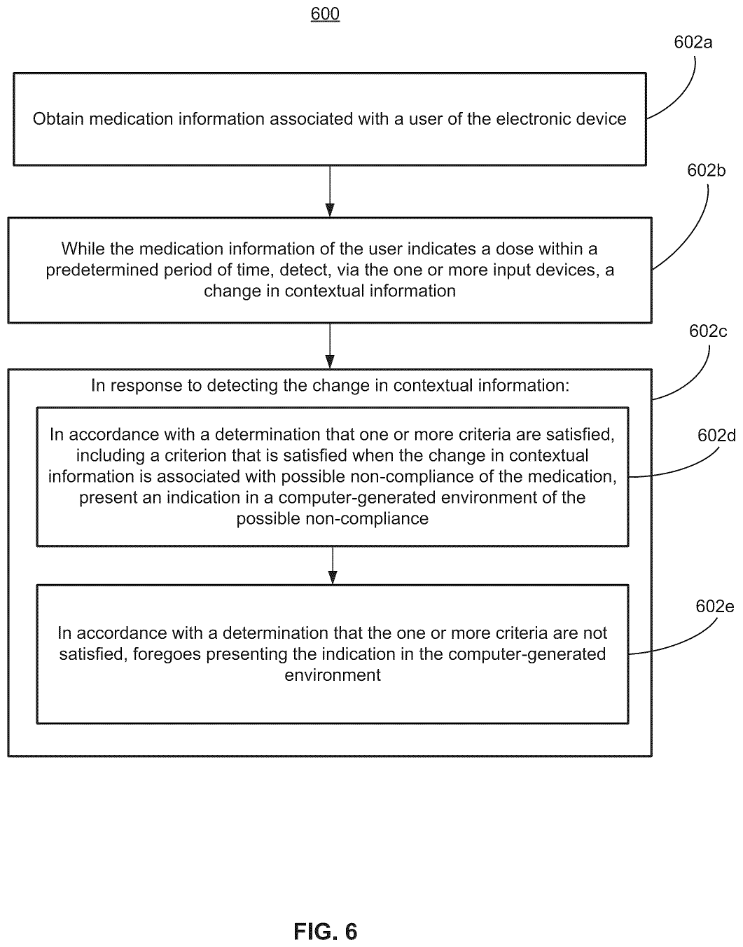

FIGS. 3A-3O illustrate examples of an electronic device displaying a representation of a prediction of a food being consumed by a user of the electronic device and an indication of possible non-compliance of medication in a computer-generated environment according to some examples of the disclosure. FIGS. 3A-3O are used to illustrate the processes described below, including the processes in FIGS. 5 and 6. The electronic device 301 may be similar to device 101 or 201 discussed above, and/or may be a head mountable system/device and/or projection-based system/device (including a hologram-based system/device) configured to generate and present a three-dimensional environment, such as, for example, heads-up displays (HUDs), head mounted displays (HMDs), windows having integrated display capability, or displays formed as lenses designed to be placed on a person's eyes (e.g., similar to contact lenses). In the example of FIGS. 3A-3O, a user is optionally wearing the electronic device 101, such that three-dimensional environment 300 (e.g., a computer-generated environment) can be defined by X, Y and Z axes as viewed from a perspective of the electronic device (e.g., a viewpoint associated with the user of the electronic device 101). Accordingly, as used herein, the electronic device 101 is configured to be movable with six degrees of freedom based on the movement of the user (e.g., the head of the user), such that the electronic device 101 may be moved in the roll direction, the pitch direction, and/or the yaw direction.

As shown in FIG. 3A the electronic device 101 presents, via a display 120, a three-dimensional environment 300 from a viewpoint of a user of the electronic device 101 (e.g., looking down at table 302 of a physical environment in which electronic device 101 is located). In some examples, a viewpoint of a user determines what content (e.g., physical and/or virtual objects) is visible in a viewport (e.g., a view of the three-dimensional environment 300 visible to the user via one or more display(s) 120, a display or a pair of display modules that provide stereoscopic content to different eyes of the same user). In some examples, the (virtual) viewport has a viewport boundary that defines an extent of the three-dimensional environment 300 that is visible to the user via the display 120 in FIGS. 3A-3O. In some examples, the region defined by the viewport boundary is smaller than a range of vision of the user in one or more dimensions (e.g., based on the range of vision of the user, size, optical properties or other physical characteristics of the one or more displays, and/or the location and/or orientation of the one or more displays relative to the eyes of the user).

In some examples, the region defined by the viewport boundary is larger than a range of vision of the user in one or more dimensions (e.g., based on the range of vision of the user, size, optical properties or other physical characteristics of the one or more displays, and/or the location and/or orientation of the one or more displays relative to the eyes of the user). The viewport and viewport boundary typically move as the one or more displays move (e.g., moving with a head of the user for a head mounted device or moving with a hand of a user for a handheld device such as a tablet or smartphone). A viewpoint of a user determines what content is visible in the viewport, a viewpoint generally specifies a location and a direction relative to the three-dimensional environment, and as the viewpoint shifts, the view of the three-dimensional environment will also shift in the viewport. For a head mounted device, a viewpoint is typically based on a location, a direction of the head, face, and/or eyes of a user to provide a view of the three-dimensional environment that is perceptually accurate and provides an immersive experience when the user is using the head-mounted device.

For a handheld or stationed device, the viewpoint shifts as the handheld or stationed device is moved and/or as a position of a user relative to the handheld or stationed device changes (e.g., a user moving toward, away from, up, down, to the right, and/or to the left of the device). For devices that include displays with video passthrough, portions of the physical environment that are visible (e.g., displayed, and/or projected) via the one or more displays are based on a field of view of one or more cameras in communication with the displays which typically move with the displays (e.g., moving with a head of the user for a head mounted device or moving with a hand of a user for a handheld device such as a tablet or smartphone) because the viewpoint of the user moves as the field of view of the one or more cameras moves (and the appearance of one or more virtual objects displayed via the one or more displays is updated based on the viewpoint of the user (e.g., displayed positions and poses of the virtual objects are updated based on the movement of the viewpoint of the user)).

For displays with optical see-through, portions of the physical environment that are visible (e.g., optically visible through one or more partially or fully transparent portions of the display generation component) via the one or more displays are based on a field of view of a user through the partially or fully transparent portion(s) of the display generation component (e.g., moving with a head of the user for a head mounted device or moving with a hand of a user for a handheld device such as a tablet or smartphone) because the viewpoint of the user moves as the field of view of the user through the partially or fully transparent portions of the displays moves (and the appearance of one or more virtual objects is updated based on the viewpoint of the user).

In FIG. 3A, the electronic device 101 includes a display 120 and a plurality of image sensors 114a as described above and controlled by the electronic device 101 to capture one or more images of a user or a part of the user (e.g., one or more hands of the user) while the user interacts with the electronic device 101. In some examples, virtual objects, virtual content, and/or user interfaces illustrated and described below could also be implemented on a head-mounted display that includes a display or display generation component that displays the virtual objects, virtual content, user interfaces or three-dimensional environment to the user, and sensors to detect the physical environment and/or movements of the user's hands (e.g., external sensors facing outwards from the user), and/or attention (e.g., including gaze) of the user (e.g., internal sensors facing inwards towards the face of the user). The figures herein illustrate a three-dimensional environment that is presented to the user by electronic device 101 (e.g., and displayed by the display 120 of electronic device 101).

As shown in FIG. 3A, the electronic device 101 captures (e.g., using external image sensors 114b and 114c) one or more images of a physical environment around electronic device 101, including one or more objects (e.g., table 302 and consumable objects 304, 306, 308, and 310) in the physical environment around the electronic device 101. In some examples, the electronic device 101 displays representations of the physical environment in the three-dimensional environment or portions of the physical environment are visible via the display 120 of electronic device 101. For example, the three-dimensional environment 300 includes consumable objects 304, 306, 308, 310, and table 302 in the physical environment.

In some examples, the electronic device 101 detects that a user of the electronic device 101 is initiating consumption of a consumable object (e.g., consumable objects 304, 306, 308, and/or 310). In some examples, consumable objects include food items, drink items, supplements, medication, and/or other substances which may be consumed (e.g., eaten and/or drunk) by the user. In some examples, prior to detecting that the user of the electronic device 101 is initiating consumption of a consumable object, the electronic device 101 determines that one or more first criteria (e.g., consumption criteria) are satisfied, including a criterion that is satisfied when a location of the electronic device 101 corresponds to a particular location (e.g., dining room, kitchen, cafeteria, restaurant, breakroom, or other location where users typically initiate consumption of a consumable object). In some examples, the one or more first criteria include a criterion that is satisfied when the electronic device 101 detects a particular posture of the user of the electronic device that is indicative of the user initiating consumption of a consumable object. For example, the posture of the user corresponds to a sitting position, a position leaning towards table 302 or other position indicative of initiating consumption of a consumable object. In some examples, the one or more first criteria include a criterion that is satisfied when the electronic device 101 detects that a time of day at the electronic device 101 is a particular time of day associated with mealtime. For example, when the electronic device 101 detects that the time of day at the electronic device 101 corresponds to a range of time (e.g., between 7:00-8:30 am, 11:30 am-2:00 pm, and/or 7:00-8:30 pm), the electronic device 101 determines that the user of the electronic device 101 is initiating consumption of a consumable object. In some examples, the electronic device determines that the one or more first criteria described above are satisfied based on data (e.g., signals) received from a subset of the one or more input devices, such as one or more location sensors 204, one or more motion and/or orientation sensors 210, and/or a clock of the electronic device 101.

In some examples, the one or more first criteria include a criterion that is satisfied when the electronic device 101 captures, using one or more input devices (e.g., external image sensors 114b and 114c) consumable objects (e.g., consumable objects 304, 306, 308, or 310) and/or physical objects indicative of initiating consumption of a consumable object in the three-dimensional environment 300 (e.g., table 302, utensils, a plate, a microwave or other physical object indicative of initiating consumption of a consumable object). In some examples, the various criteria described above may be based on learned trends from historical data collections. For example, if the user of the electronic device 101 typically washes their hands before initiating consumption of a consumable object, the electronic device 101 may be determine that the one or more criteria are satisfied after capturing, using one or more input devices (e.g., external image sensors 114b and 114c), a combination of hand movements associated with washing the user's hands and/or the presence of water and/or hand soap. In another example, if the user of the electronic device 101 typically interacts with a secondary device, such as mobile phone or tablet, to watch a television show, listen to a podcast, or other interaction with their secondary device when initiating consumption of a consumable object, the electronic device 101 may determine that the one or more criteria are satisfied after detecting actuation of a physical input device of or in communication with the secondary device. In some examples, the one or more first criteria include a criterion that is satisfied when the electronic device 101 detects user input indicative of initiating consumption of a consumable object, such as a gaze-based input corresponding to the consumable object, audio input associated with the consumable object (e.g., food packaging being opened), a predefined gesture associated with the consumable object (e.g., picking up the consumable object) and/or a voice input from the user associated with the consumable object (e.g., spoken food-related keywords or phrases, such as “Let's eat”, “I'm hungry”, “I'm thirsty”, “What's for lunch?” and/or the like).

In some examples, in accordance with a determination that the one or more criteria are not satisfied (e.g., the location of the electronic device 101 does not correspond to a kitchen or dining area), the electronic device 101 does not detect that the user of the electronic device 101 is initiating consumption of a first object. In some examples, the electronic device 101 does not initiate capturing, using one or more input devices (e.g., external image sensors 114b and 114c), audio and/or imagery of the three-dimensional environment 300 as indicated by microphone indicator 312 and image sensor indicator 314 remaining and/or being deactivated in FIG. 3A. In some examples, the electronic device 101 does not initiate an image analyzer mode as indicated by image analyzer mode indicator 316 with value “OFF”, as will be described in more detail below with reference to FIG. 3D.

As shown in FIG. 3B, the electronic device 101 determines that the user of the electronic device 101 is initiating consumption of a first object. For example, as described above, the electronic device 101 determines that one or more consumption criteria are satisfied, and in response to determining that the user of the electronic device is initiating consumption of the first object, the electronic device 101 captures, using the one or more input devices, audio (e.g., sound print 318) of the consumption of the first object (e.g., consumable object 310). In some examples, the electronic device 101 captures the audio of the consumption of the first object using the one or more input devices as indicated by microphone indicator 312 in FIG. 3B. In some examples, the electronic device 101 captures the audio of the consumption of the first object in accordance with a determination that the electronic device is initiating consumption of the first object as described above. In some examples, the electronic device 101 captures the audio of the consumption of the first object in accordance with a determination that the one or more first criteria (e.g., consumption criteria) are satisfied, including a criterion that is satisfied when a pose or posture of the user of the electronic device 101, using one or more motion and/or orientation sensors 210, indicates a consumption (eating or drinking) position (e.g., hand of user holding the first object and/or is within a predetermined distance (e.g., 0, 1, 2, 3, 5, 10, 15, 20, 25, 30, or 50 cm) from the user's mouth, and/or the head of the user is in a particular position/orientation leaning towards the first object)). In some examples, the electronic device 101 determines that the captured audio satisfies one or more audio consumption criteria including a criterion that is satisfied when the audio captured corresponds to chewing and/or drinking sounds for more than a threshold period of time (e.g., 5, 10, 20, 30, 40, 50, or 60 seconds). In some examples, the electronic device 101 confirms the audio captured indicates consumption of the first object by triggering acquisition and processing of image data, such as when imagery acquired and processed via internal sensors facing inwards towards the face of the user indicate facial expressions that reflect the user chewing and/or drinking. In some examples, the electronic device 101 confirms consumption of the first object when the first object is within a predetermined distance (e.g., 0, 1, 3, 5, 10, 20, 30, 40, 50, 60, 70, 80, 90, 100, 125 or 150 cm) from the user of the electronic device 101. In the example of FIG. 3B, the electronic device 101 determines that consumable object 310 is within the predetermined distance from the user of the electronic device 101, thereby confirming consumption of the first object.

In some examples, after the electronic device 101 captures the audio and (optionally the one or more images of the first object as described herein), the electronic device 101 obtains a first prediction of the first object based on a sound print of the first object included in the audio. For example, in FIG. 3C, the electronic device 101 outputs a first prediction (e.g., representation 320a), “Iced Drink” based on the sound print 318 of the first object (e.g., consumable object 310) included in the captured audio. In some examples, the electronic device 101 displays representation 320a in the three-dimensional environment 300 via the display 120. In some examples, the electronic device 101 saves the sound print 318 and first prediction of the first object to a remote server/database and/or a local database (e.g., maintained by electronic device 101 from an application operating on the electronic device 101 and/or by a third-party in communication with the electronic device 101). Alternatively, in some examples, the electronic device 101 foregoes displaying the representation 320a via the display 120 (but still saves the sound print 318 and the first prediction of the first object).

In some examples, obtaining the first prediction (e.g., illustrated via representation 320a) of the first object (e.g., consumable object 310) based on the sound print 318 of the first object included in the audio includes the electronic device 101 identifying an object from a plurality of objects that has a respective sound print that matches the sound print of the first object when a score of the object is within a predetermined score. For example, the electronic device 101 transmits the sound print 318 of the first object included in the captured audio and optionally the one or more captured images of the first object for look-up in the remote server/database and/or the local database. In some examples, the electronic device 101 searches for a substantially matching sound print within a database of sounds prints and object pairs. For example, the first prediction corresponds to a first match of the sound print 318 and a known sound print of a respective object. In some examples, the first prediction is selected because a score of the first match is within a predetermined score range (e.g., between 60 and 100 points) or greater than a score threshold. In some examples, the score indicates a probability that the same sound print describes (e.g., is a match for) at least two objects. In this case, and in some examples, the electronic device 101 measures the score against a threshold. If the score is below the threshold, the electronic device 101 initiates a process to analyze the one or more images of the first object to identify the first object unambiguously as will be described below. In some examples, if the score meets or exceeds the predetermined threshold, then the electronic device 101 foregoes initiating the process to analyze the one or more images of the first object and the respective prediction is saved to the database. In some examples, the electronic device 101 saves the respective prediction and the sound print as a sound print and object pair in the database, such that the respective prediction is associated with the sound print.

In FIG. 3C, the first prediction (e.g., illustrated via representation 320a) of the first object (e.g., consumable object 310) is associated with a respective score, as represented by confidence indicator 320b. In the example of FIG. 3C, the electronic device 101 determines that the respective score satisfies one or more prediction criteria, including a criterion that is satisfied when a score associated with a respective prediction is below a predetermined threshold 320c. In this instance, because the respective score associated with the first prediction satisfies the one or more predication criteria (e.g., is below the predetermined threshold 320c), the electronic device 101 determines that the first prediction is ambiguous. Particularly, the first prediction “Iced Drink” could be interpreted as corresponding to different iced drinks (e.g., iced coffee, iced soda, iced water, iced juice, etc.) and that a better match for the first object may be determined by analyzing additional data. For example, the electronic device 101 initiates a process to analyze the one or more captured images of the first object as indicated in FIG. 3D with the image analyzer mode indicator 316 with value “ON”.

In some examples, initiating a process to analyze the one or more captured images of the first object includes identifying a plurality of objects included in the one or more images. For example, the electronic device 101 may apply computer vision processing, optical character recognition (OCR), or other recognition technique to detect and/or identify the plurality of objects. In some examples, the electronic device 101 transmits the one or more captured images of the three-dimensional environment 300 for look-up in the remote server/database and/or the local database discussed above to identify the plurality of objects in the captured images (e.g., table 302 and consumable objects 304, 306, 308, and 310). In FIG. 3D, the electronic device 101 obtains a second prediction of the first object (e.g., illustrated via representation 322a) based on one or more of the plurality of objects included in the one or more images (e.g., identified objects corresponding to table 302 and consumable objects 304, 306, 308, and 310). In some examples, the electronic device 101 searches for a substantially matching image or image feature within a database of images and object pairs. For example, the second prediction corresponds to a first match of the image data and a respective object. In some examples, the second prediction is selected because a score of the first match is within the predetermined score range or greater than a threshold score as described above. In FIG. 3D, the second prediction (e.g., representation 322a) of the first object (e.g., consumable object 310) is associated with a respective score as represented by confidence indicator 322b that does not satisfy the one or more prediction criteria as described above (e.g., the score meets or exceeds the predetermined threshold, represented by threshold 322c). In this instance, the second prediction is saved to the database. In some examples, the electronic device 101 saves the second prediction and the image data as an image data and object pair in the database such that the second prediction is associated with the image data.

In some examples, and as shown in FIG. 3D, the electronic device 101 presents, via the display 120, a user interface 324a via which a user of the electronic device 101 may confirm or deny that the second prediction is correct. For example, as shown in FIG. 3D, the user interface 324a includes options 324b and 324c that are selectable to confirm or deny, respectively, that the second predication is correct. In FIG. 3D, the electronic device 101 detects a pinch gesture from hand 328 from the user of the electronic device 101, optionally while gaze 326 of the user of the electronic device 101 is directed towards option 324, confirming that the second prediction is correct. In some examples, the electronic device 101 presents user interface 324a while the electronic device 101 detects consumption of the first object and/or after consumption of the first object is complete as determined using the one or more captured images of the three-dimensional environment 300. In some examples, the electronic device 101 presents user interface 324a in accordance with a determination that the score associated with the second prediction satisfies the one or more prediction criteria as described above (e.g., the score meets or exceeds the predetermined threshold indicative of potential ambiguity).

In some examples, obtaining the second prediction of the first object (e.g., illustrated via representation 322a) includes identifying a second object of the plurality of objects included in the one or more images which has a respective sound print that matches the sound print of the first object when a score of the second object is within a predetermined score. For example, based on the image data analysis, the electronic device 101 identifies consumable object 310 (e.g., “Iced Coffee”). In some examples, the electronic device 101 transmits data describing consumable object 310 for look-up in the remote server/database and/or the local database to identify a corresponding sound print. In some examples, the electronic device 101 compares the corresponding sound print with the sound print of the first object (e.g., sound print 318) and if the sounds prints substantially match because a score of the match is within the predetermined score range as described above, the electronic device 101 saves the sound print of the first object (e.g., sound print 318) in the database such that the respective object is associated with this newly captured sound print (e.g., sound print 318).

As discussed above, the first prediction or the second prediction is optionally saved to the database that is remote or local to the electronic device 101. In some examples, the electronic device 101 automatically adds and/or saves data corresponding to the first prediction or the second prediction to a digital journal accessible on the electronic device 101. For example, in FIG. 3E, the electronic device 101 displays, via the display 120, an indication (e.g., representation 330) that the second prediction (e.g., representation 322a) is saved to the digital journal. In some examples, the electronic device 101 foregoes displaying the representation 322a via the display 120. In some examples, if the electronic device 101 determines that the respective score associated with the prediction is outside the predetermined score described above, the electronic device 101 does not save the prediction to the database and/or does not display a representation of the prediction. In some examples, if the electronic device 101 determines that the respective score does not meet or exceed the predetermined threshold described above, the electronic device 101 does not save the prediction to the database and/or does not display a representation of the prediction. In some examples, prior to saving the first prediction or the second prediction to the database, the electronic device 101 displays, via the display 120, a user interface in the three-dimensional environment 300 via which the user of the electronic device 101 may accept or decline saving the data corresponding to the first prediction or the second prediction to the digital journal. For examples the user interface may be similar to user interface 324a and includes options that are selectable to accept or decline, respectively, saving the data corresponding to the first prediction or the second prediction to the digital journal.

In FIG. 3F, the electronic device 101 detects that the user of the electronic device 101 is initiating consumption of a second object (e.g., different from the first object) and in response to detecting that the user of the electronic device 101 is initiating consumption of the second object, the electronic device 101 captures, using the one or more input devices (e.g., external image sensors 114b and 114c), audio (e.g., sound print 334) and one or more images of the second object (e.g., consumable object 332). In some examples, the electronic device 101 captures the audio and the one or more images of the second object using the one or more input devices, as indicated by microphone indicator 312 and image sensor indicator 314 being activated in FIG. 3F.

In some examples, the electronic device 101 obtains a first prediction of the second object based on a sound print of the second object included in the audio. For example, in FIG. 3G, the electronic device 101 outputs a first prediction (e.g., representation 336a) “Chips” based on the sound print 334 of the second object (e.g., consumable object 332) included in the captured audio. In some examples, the electronic device 101 displays representation 336a via the display 120. In some examples, the electronic device 101 saves the sound print 334 and first prediction of the second object to the remote server/database and/or the local database. In some examples, the electronic device 101 adds and/or saves the first prediction to the digital journal accessible on the electronic device 101. For example, in FIG. 3H, the electronic device 101 displays, via the display 120, an indication (e.g., representation 338) that the second prediction (e.g., representation 336a) is saved to the digital journal. Alternatively, in some examples, the electronic device 101 foregoes displaying the representation 336a in the three-dimensional environment 300 via the display 120 (but still adds and/or saves the first prediction to the remote server/database and/or the local database and/or the digital journal).

In some examples, power consumption can be reduced by implementing one or more power saving mitigations. For example, power saving mitigations optionally include ceasing image data acquisition and/or analysis in response to a determination that the first prediction (e.g., representation 336a in FIG. 3G) based on the sound print 334 of the second object (e.g., consumable object 332) included in the captured audio is associated with a respective score as represented by confidence indicator 336b that does satisfy the one or more prediction criteria as described above (e.g., the score meets or exceeds the predetermined threshold, represented by threshold 336c). In this instance, the electronic device 101, in response to the determination that the respective score satisfies the one or more prediction criteria, foregoes and/or ceases processing image data because the electronic device is confident that the sound print 334 included in the captured audio corresponds to the first prediction “Chips”. In some examples, acquiring and analyzing the audio data can be used to trigger the acquisition and/or analysis of image data. In some examples, processing of audio data includes lower processing power than the processing power used for the acquisition and/or processing of image data and thus, the first prediction and associated respective confidence score is optionally used to trigger the acquisition and/or processing of image data.

In some examples, the electronic device 101 displays, via the display 120, a user interface of the digital journal, such as user interface 340a in FIG. 3I. In some examples, the digital journal is a module of a journaling application or a health application configured to provide a user's health information including a log or history of foods, drinks, and/or medications consumed by the user as captured and identified by the electronic device 101 and/or provided by the user. In some examples, the user interface 340a includes nutrition insights and/or trends as described below.

In some examples, the electronic device 101 displays user interface 340a in response to user interaction corresponding to a request to display the user interface 340a. In some examples, user interaction includes: a gaze of the user; a finger of a hand and/or the hand touching, grabbing, holding a physical object; a finger of the hand directed to or within a threshold distance (e.g., 0, 1, 2, 3, 5, 10, 15, 20, 25, 30, or 50 cm) from a location corresponding to a virtual object selectable to display user interface 340a; a finger of the hand touching physical buttons of the electronic device 101; a contact on a touch-sensitive surface; actuation of a physical input device; a predefined gesture, such as a pinch gesture or air tap gesture; and/or a voice input from the user of the electronic device 101 corresponding to the request to display user interface 340a. In some examples, the electronic device 101 displays user interface 340a in response to satisfying one or more conditions. For example, satisfaction of the one or more conditions is based on a predetermined date and/or time (e.g., morning time, end of the day at 5:30 pm, end of the week, or end of the month) and/or a detected event (e.g., before a grocery shopping event, or at the start or end of an eating episode). In some examples, the electronic device 101 displays user interface 340a in response to adding and/or saving data to a digital journal corresponding to a prediction of a food being consumed by the user as described above.

In some examples and as illustrated in FIG. 3I, user interface 340a includes a log of foods and/or drinks consumed by a user of the electronic device 101 over a period of time (e.g., for a particular day, such as Wednesday, August 14). In some examples, the period of time alternatively spans days, weeks, months, or years. In FIG. 3I, the user interface 340a includes a representation of journal entry 340b of logged data for a first consumption episode (e.g., “Breakfast”) and a representation of journal entry 340c of the logged data for a second consumption episode (e.g., “Lunch”). Journal entry 340b optionally includes content related to a location of the electronic device at which the first consumption episode occurred (e.g., “Home”) and a time representing the start of the first consumption episode (e.g., “8:15 am”). Similarly, journal entry 340c optionally includes content related to a location of the electronic device at which the second consumption episode occurred (e.g., “Office”) and a time representing the start of the first consumption episode (e.g., “12:05 pm”). In some examples, the location of the electronic device 101 at which the consumption episode occurred is obtained from a maps or navigation application or from a calendar application on the electronic device 101. In some examples, journal entries 340b and 340c include a listing of foods and/or drinks consumed by the user of the electronic device during the first consumption episode and the second consumption episode, respectively. In some examples, each of the foods and/or drinks listed is selectable to cause the electronic device to present detailed information about the selected food and/or drink, such as information related to nutrition, portion size, and/or user trends. For example, in response to detecting user interaction corresponding to a request to display detailed information related to the “Water” item of journal entry 340c (e.g., a selection of the Water item), the electronic device 101 displays user interface element 340g that includes a graphical representation illustrating an average amount of water the user of the electronic device 101 consumes per day, as indicated by the value “7” displayed by user interface element 340g. In some examples, the electronic device 101 automatically displays the user interface element 340g (or a similar user interface element for another food or drink item) concurrently with user interface 340a (e.g., without first receiving the user request to display user interface element 340g).

In some examples, a journal entry includes information related to the respective consumption episode, such as environmental contextual information and user state information. Environmental contextual information and user state information optionally describe one or more detected activities of the user during the respective consumption episode (e.g., watching television, reading emails, consuming media content, talking on the phone, interacting with a secondary device, different from electronic device 101, and/or the like). Environmental contextual information and user state information optionally include indications of other users detected during the respective consumption episode (e.g., users physically present in the environment surrounding electronic device 101 or users present in a remote or virtual manner). For example, the electronic device 101 detects, using one or more input devices (e.g., external image sensors 114b and 114c) users physically present in the three-dimensional environment 300 who are within the user's field of view and/or are within a predetermined distance (e.g., 50, 100, 150, 200, 250, 300, 350, 400, 450, or 500 cm) from the user of the electronic device 101. In another example, the electronic device 101 determines that the user of the electronic device 101 is interacting with remote users virtually based on engagement with a video telephony application, videoconference application, and/or the like on the electronic device 101. In some examples, environmental contextual information and user state information include the mood or physiological condition of the user of the electronic device during the respective consumption episode as determined through detected user data related to the user's heart rate, eye gaze, tone of voice, breathing, temperature, and/or posture. In some examples, the above environmental contextual information and user state information related to the respective consumption episode may be utilized by the electronic device 101 to generate trends and/or other user characteristics, such as, for example, the likelihood the user consumes a particular type of food, an amount of a particular food, time and/or location of food intake, and/or the like. For example, if the user always consumes food and/or drink that is high in added sugar when the user is in a stressed user state, the electronic device 101 may determine that the user will likely consume food and/or drink with added sugar after detecting a combination of physiological characteristics (e.g., high heart rate and/or change in tone of voice) and/or other historical data, and in response, the electronic device 101 may notify the user of such trend and optionally recommend different foods and/or drinks that are healthier than the food and/or drink with added sugar. In another example, if the user always consumes food and/or drink that is high in added sugar when the user is engaged in a particular activity, such as watching television, the electronic device 101 may determine that the user will likely consume food and/or drink with added sugar after detecting activation of the television and in response, the electronic device 101 may notify the user of said determined trend and optionally recommend different foods and/or drinks that are healthier than the food and/or drink with added sugar (e.g., foods with lower sugar or no added sugar).

In some examples, the electronic device 101 may be configured to display, via the display 120, content related to a user's medication regime (e.g., reminders to the user of the electronic device 101 to take their medication). For example, in FIG. 3I, the user interface 340a includes content 340d related to the user's medication regime. In some examples, content 340d serves as a reminder to the user to consume their medication. In some examples, the electronic device 101 presents content 340d at a determined time (e.g., optional time for taking their medication) as will be described in more detail below. In FIG. 3I, content 340d includes information related to the medication, such as the name, an amount, and a time to take the medication. In some examples, content 340d optionally includes an option that when selected, causes the electronic device 101 to log that the medication has been taken by the user. In some examples, the electronic device 101 may automatically log successful consumption of the medication in response to detection that the user consumed the medication (e.g., as similarly discussed above). In some examples and as will be described in more detail with reference to FIGS. 3J to 30, the electronic device 101 monitors for contextual information (e.g., user and/or environmental context data) that may indicate possible non-compliance of taking the medication and/or a favorable time to prompt the user to consume the medication. For example, the electronic device 101 determines, based on a context of the user and/or the environment of the user, a likelihood of compliance with a medication's prescribed medicinal regimen in order to notify the user when medication is being taken in a way that differs from the prescribed medicinal regimen.

In some examples, the user of the electronic device 101 may have a medication profile that is accessible and/or stored by the electronic device 101. For example, the user of the electronic device 101 can create or update a medication profile for storage by the electronic device 101 (e.g., by the remote server/database and/or local database described above). In some examples, and as shown in FIG. 3J, the user of the electronic device 101 has provided medication information via user interface 342a configured to capture the user's medication information. In some examples, user interface 342a is a user interface of the health application as described above. In some examples, the medication information captured by user interface 342a includes a name or other indicator of the prescription medication (e.g., representation 342b), a description and/or amount of the prescription medication (e.g., representation 342c), and a prescribed timing (e.g., “everyday at 10:00 am”) as indicated by representation 342d. In some examples, the user interface 342a includes option 342e that, when selected, causes the electronic device 101 to store the medication information to the database (e.g., the user's medication profile). In some examples, the medication information may be automatically entered by a prescription management system, a medication provider application, and/or the like in communication with the electronic device 101. In some examples, the medication information may be determined automatically by the electronic device 101 based on automatically retrieved data from the prescription management system, the medication provider application, or other external information source (e.g., a care provider's medical records).

In some examples, upon receipt of the medication information via user interface 342a, the electronic device 101 may look up, in the above-mentioned databases, information about the medication, such as side effects, interactions, precautions, and/or best use. In some examples, the electronic device 101 may assign or associate the medication to a specification or strategy used as a reference in order to provide notifications to the user related to side effects, interactions, precautions, and/or best use. The notifications optionally include content related to possible non-compliance of taking the medication and/or are presented to the user automatically at favorable times given contextual information, as discussed below.

In some examples, the electronic device 101 initiates the collection of contextual information from the various sensors described herein in accordance with a determination that the medication information indicates a prescribed or recommended dose within a predetermined period of time (e.g., 0.5, 3, 6, 12, or 24 hours). In some examples, the electronic device 101 begins collecting data from the various sensors after the user has already consumed the medication. For example, some medications require a period of rest before the user engages in activities such as consuming foods or exercising. In some examples, the electronic device 101 does not begin collecting data from the various sensors if the current time is outside the predetermined period of time associated with consumption of the medication. In some examples, the electronic device 101 does not begin collecting data from the various sensors if the medication has not yet been consumed.

In some examples, while the medication information of the user indicates a dose within the predetermined period of time described above, the electronic device 101 detects, via the one or more input devices, a change in contextual information. For example, the change in contextual information relates to detected activity of the user during the predetermined period of time of the dose. Detected activity optionally include the user exercising, eating, drinking, consuming other medication, shopping, interactions with machinery, and/or the like. In some examples, the change in contextual information relates to one or more physical characteristics of the user during the predetermined period of time of the dose, such as for example, the user's heart rate, breathing pattern, temperature, eye gaze, and/or posture. In some examples, the change in contextual information relates to location information corresponding to a physical environment of the user of the electronic device during the predetermined period of time of the dose. In some examples, the change in contextual information corresponding to the physical environment includes changes in the physical location, temperature, amount of sun exposure, and/or the like.

In some examples, in response to detecting the change in contextual information, the electronic device 101 determines whether one or more medication criteria are satisfied, including a criterion that is satisfied when the change in contextual information is associated with possible non-compliance of the medication. For example, in FIG. 3K, the electronic device 101 detects that the user is interacting with a user interface 344a of a web browser or a shopping application. In some examples, while the electronic device 101 detects that the user is interacting with a user interface 344a, the electronic device 101 determines that the user is searching for wine, as shown by user interface search element 344b (e.g., search field including text “wine”) and/or search results 344c. In some examples, the electronic device 101 determines that wine or any alcohol is associated with possible non-compliance of the medication, and in response to determining that the wine is associated with possible non-compliance of the medication, the electronic device 101 presents an indication in the three-dimensional environment 300 of the possible non-compliance as illustrated in FIG. 3L via representation 346. In some examples, representation 346 is a user interface notification including information that the consumption of wine may result in non-compliance of the medication. In some examples, representation 346 includes information corresponding to a recommendation or alternative to wine, such as sparkling juice. In some examples, the electronic device 101 presents recommendations that satisfy search parameter(s) of the user's search query for wine. In such example, the recommended “sparkling juice” satisfies search parameters “sparkling”, “bubbles”, “celebration”, and/or “sweet”. In some examples, representation 346 includes information indicative of the predetermined period of time of the dose and/or timing when consuming wine is not associated with possible non-compliance (e.g., “Aug. 25, 2023”). In some examples, the information provided by representation 346 is derived from the medication specification described above.

In some examples, while presenting the indication of the possible non-compliance (e.g., representation 346), the electronic device 101 detects, via the one or more input devices (e.g., image sensors 114a, 114b, and/or 114c), a second change in contextual information. For example, in FIG. 3M, the electronic device 101 detects that the user is interacting with the user interface 344a. In some examples, while the electronic device 101 detects that the user is interacting with the user interface 344a, the electronic device 101 determines that the user is searching for sparkling juice, as shown by user interface search element 344d and/or search results 344e. In some examples, the electronic device 101 determines that sparkling juice is not associated with possible non-compliance of the medication (e.g., the one or more criteria described above are not satisfied), and in response to determining that the sparkling juice is not associated with possible non-compliance of the medication, the electronic device 101 ceases presentation of the indication (e.g., representation 346 of FIG. 3L) in the three-dimensional environment 300. In some examples, if the electronic device 101 determines that the one or more criteria are satisfied, including the criterion that is satisfied when the second change in contextual information is associated with possible non-compliance of the medication, the electronic device 101 maintains presentation of the indication (e.g., representation 346 of FIG. 3L) in the three-dimensional environment 300.

As mentioned above, the electronic device 101 may determine a favorable time to prompt the user of the electronic device 101 to take their medication. For example, the electronic device 101 determines that a current time of day at the electronic device 101 is within the predetermined period of time of a dose of the medication, and in response to the determination that the current time of day at the electronic device 101 is within the predetermined period of time of the dose of the medication, the electronic device 101 presents an indication in the three-dimensional environment 300 prompting the user of the electronic device 101 to initiate consumption of the medication. For example, FIG. 3N illustrates an exemplary indication 348a. In some examples, indication 348a is a notification automatically generated and displayed in response to the determination that the current time of day at the electronic device 101 is within the predetermined period of time of the dose of the medication. In some examples, indication 348a includes the prescription medication description and/or amount of the prescription medication (e.g., representation 348b), and a prescribed timing (e.g., “everyday at 8:15 am”) as indicated by representation 348c. In some examples, if the electronic device 101 determines that one or more criteria are not satisfied (e.g., the current time of day at the electronic device 101 is outside the predetermined period of time of the dose of the medication), the electronic device 101 forgoes presenting indication 348a. In some examples, if the electronic device 101 detects, via the one or more input devices (e.g., internal sensors facing inwards towards the face of the user), that the user has consumed the medication, the electronic device 101 ceases to display indication 348a in the three-dimensional environment 300.

In some examples, the favorable time to prompt the user of the electronic device 101 to take their medication may be based on a location of the user of the electronic device 101 (e.g., and thus the location of the electronic device 101). For example, some medications are better absorbed when taken with food. Accordingly, as shown in FIG. 3N, when the electronic device 101 determines that the user is located in a kitchen area as determined by objects in the field of view of the user in the three-dimensional environment 300 (e.g., coffee maker 350, coffee cup, and/or kitchen counter 354), the electronic device 101 presents indication 348a. In another example, the favorable time to prompt the user of the electronic device 101 to take their medication may be based on a schedule of the user of the electronic device 101. For example, the electronic device 101 may have access to the user's calendar application and may determine that the user will be in a meeting or will be engaged in a scheduled activity for certain blocks of time. In this case, the electronic device 101 may prompt the user of the electronic device 101 to take their medication at a different time (e.g., before or after 8:15 AM) that is outside the user's scheduled calendar activities, but that is still within the predetermined period of time of the prescribed timing of the medication.

In some examples, the favorable time to prompt the user of the electronic device 101 to take their medication may be based on user state information, such as the mood or physiological condition of the user of the electronic. In some examples, the electronic device 101 detects that the change in contextual information relates to the user state. For example, in FIG. 3O, the electronic device 101 detects that the heart rate of the user has increased as indicated by the increased heart rate value indicator 346 in FIG. 3O compared to the heart rate value indicator 346 in FIG. 3N. In some examples, the electronic device 101 determines that the change in contextual information (e.g., the increased heart rate of the user) is associated with possible non-compliance of the medication, and in response to the determination that the change in contextual information is associated with possible non-compliance of the medication, the electronic device 101 presents a second indication 356, different from indication 348a. that includes content of the possible non-compliance. For example, indication 356 includes content informing the user of their increased heart rate and to consider lowering their heart rate before consuming their medication. In some examples, in response to the determination that the change in contextual information is associated with possible non-compliance of the medication, the electronic device 101 ceases to display indication 348a as described above. In some examples, if the electronic device 101 determines that the heart rate of the user is lowered to a level that is not associated with non-compliance of the medication, the electronic device 101 ceases to display indication 356 (and optionally redisplays the indication 348a discussed above).

Accordingly, various examples for displaying representations of predictions of foods being consumed by a user of an electronic device and in turn, saving the foods consumed and providing trends related to the foods consumed in a digital journal enables a user to keep track and view information about foods consumed by the user, thereby simplifying the presentation of information to the user and interactions with the user, which enhances the operability of the device and makes the user-device interface more efficient (e.g., by helping the user to provide proper inputs and reducing user mistakes when operating/interacting with the device), which, additionally, reduces power usage and improves battery life of the device by enabling the user to use the device more quickly and efficiently. The electronic device may additionally provide indications of possible non-compliance of medication. These indications may be generated based, at least in part, on contextual information including a determined state of the user and/or the environment of the user of the electronic device. Moreover, the indications provide the user with information regarding the potential impact of their actions and/or the environment on the user's medicinal therapy.

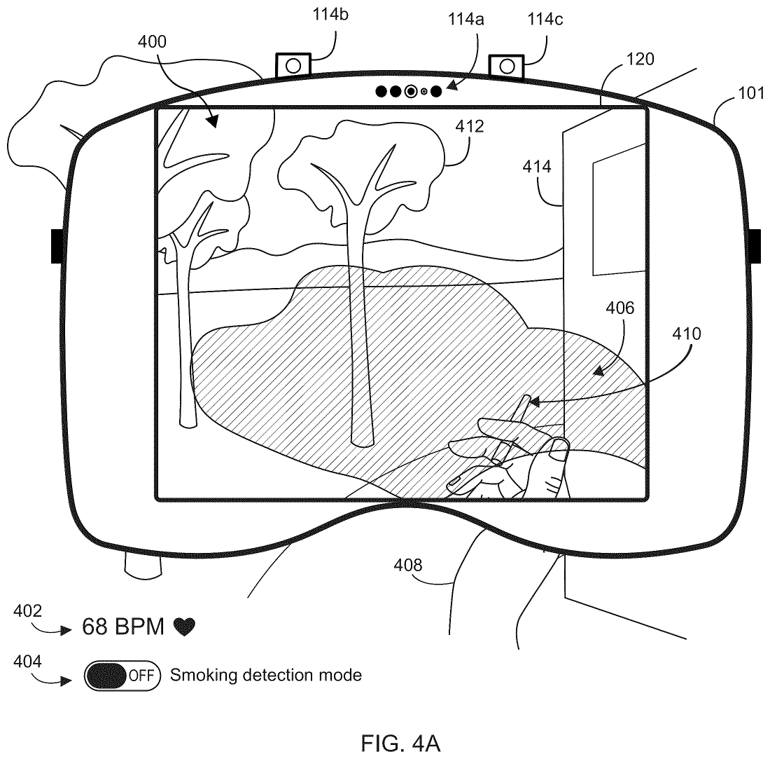

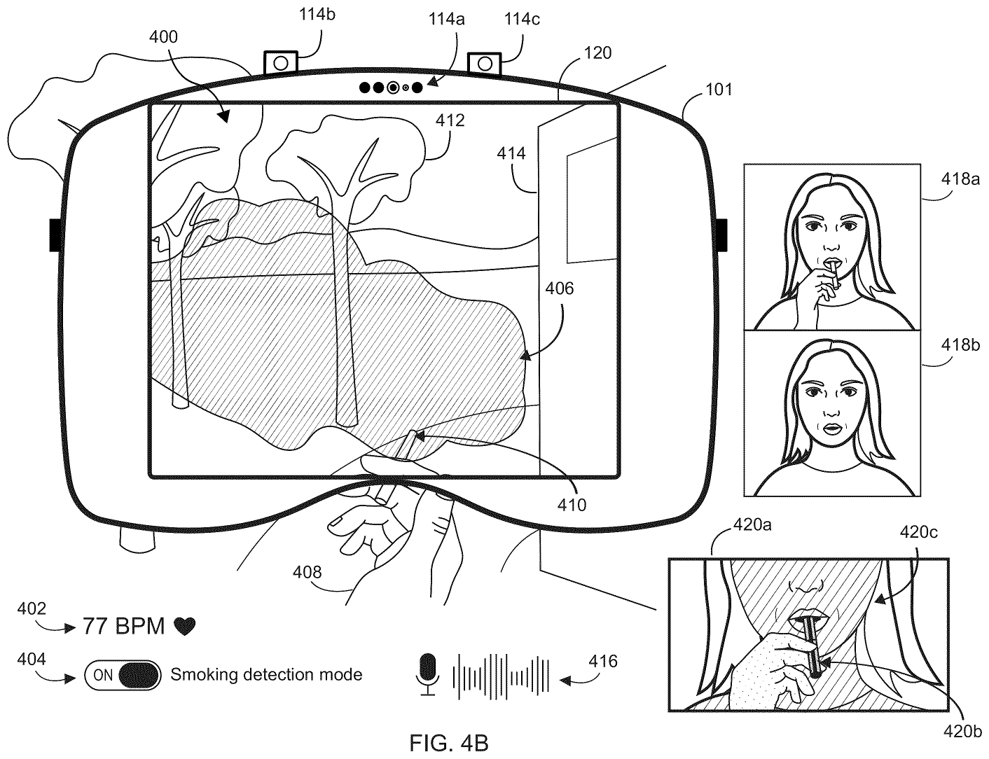

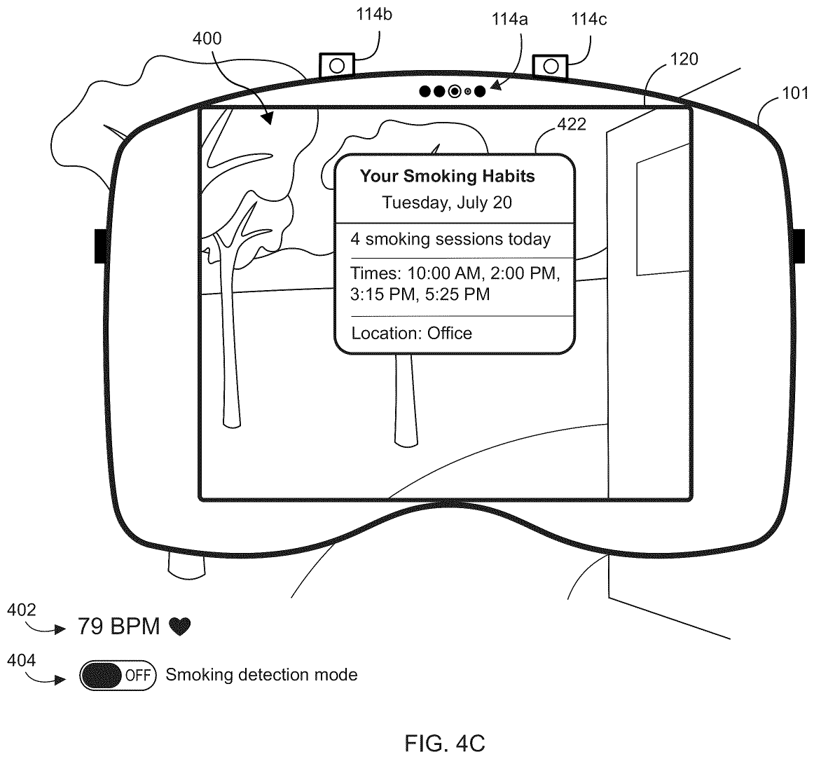

Attention is now directed to examples of displaying user interfaces based on an electronic device initiating a smoking detection mode according to some examples of the disclosure. FIGS. 4A-4C illustrate examples of an electronic device initiating a smoking detection mode according to some examples of the disclosure. As described above electronic device 101 may be a head mountable system/device and/or projection-based system/device (including a hologram-based system/device) configured to generate and present a three-dimensional environment, such as, for example, heads-up displays (HUDs), head mounted displays (HMDs), windows having integrated display capability, or displays formed as lenses designed to be placed on a person's eyes (e.g., similar to contact lenses). In some examples, as shown in FIG. 4A, the electronic device 101 is presenting a three-dimensional environment 400 (e.g., a computer-generated environment) viewed from a perspective of the electronic device (e.g., a viewpoint associated with the user of the electronic device 101).

As shown in FIG. 4A, the electronic device 101 may be positioned in a physical environment (e.g., an outdoors environment) that includes a plurality of real-world objects. For example, in FIG. 4A, the electronic device 101 may be positioned in a physical environment that includes a building 414, sidewalks, trees 412, streetlamps, and/or the like (e.g., the user of the electronic device 101 is standing in the physical environment). Accordingly, in some examples, the three-dimensional environment 400 presented using the electronic device 101 optionally includes captured portions of the physical environment surrounding the electronic device 101, such as one or more representations of building 414 in the field of view of the three-dimensional environment 400. In some examples, the representations can include portions of the physical environment viewed through a transparent or translucent display of electronic device 101 as described herein. In some examples, the three-dimensional environment 400 has one or more characteristics of the three-dimensional environment 300 discussed above.

In FIG. 4A, the electronic device 101 detects, via one or more input devices (e.g., external image sensors 114b and 114c), smoke in the physical environment without detecting a visual indication that the user of the electronic device 101 is smoking. In some examples, detecting the visual indication that the user of the electronic device 101 is smoking includes detecting a smoking instrument, such as a cigarette, cigar, pipe, e-cigarette, and/or the like. In some examples, the smoke in the physical environment may obscure the visibility of the smoking instrument such that the electronic device 101 is unable to detect the smoking instrument (e.g., via the external image sensors 114b and 144c). For example, in FIG. 4A, the electronic device 101 detects smoke 406 in the physical environment (e.g., wherein the smoke 406 is obscuring cigarette 410 that is held by the hand 408 of the user of the electronic device 101).

In some examples, and as described below with reference to FIGS. 4B and 4C, the electronic device 101 may obtain and analyze other data from the user of the electronic device 101 and/or from the physical environment to confirm whether the user of the electronic device 101 is smoking. In some examples, once the electronic device 101 confirms that the user is smoking, the electronic device 101 initiates a smoking detection mode to log information about the smoking event to help inform the user of their smoking habits. For example, in response to detecting the smoke 406 in the physical environment without detecting the visual indication that the user of the electronic device is smoking as discussed above, the electronic device 101 captures one or more images of the smoke 406 and transmits the one or more images of the smoke 406 for comparison against a database of smoking data including smoke patterns indicative of smoking. In some examples, when the electronic device 101 determines the smoke 406 matches a known smoke pattern, the electronic device initiates activation of the smoking detection mode as indicted by the smoking detection mode indicator turned from “OFF” in FIG. 4A to “ON” in FIG. 4B.

In some examples, other data from the user may indicate the user is smoking. For example, in FIG. 4B, the electronic device detects that the heart rate of the user has increased as indicated by the increased heart rate value indicator 402 in FIG. 4B compared to the heart rate value indicator 402 in FIG. 4A (e.g., has increased from 68 BPM to 77 BPM). In some examples, the electronic device 101 determines that the user state information (e.g., the increased heart rate of the user) is associated with smoking, and in response to the increased heart rate of the user in combination with the smoke 406, the electronic device 101 initiates activation of the smoking detection mode. In some examples, the electronic device 101 analyzes other data to further confirm or deny that the user is smoking. For example, the electronic device 101 captures audio from the three-dimensional environment 400 (e.g., the physical environment included in the three-dimensional environment 400) including audio from the user of the electronic device 101. In some examples, a sound print 416 from the captured audio is transmitted to the database of smoking data including audio patterns indicative of smoking. In some examples, when the electronic device 101 determines the sound print 416 matches a known audio pattern indicative of smoking, the electronic device 101 further confirms that the user is indeed smoking and initiates activation of the smoking detection mode.

In yet another example, if the electronic device 101 detects movement and/or pose of the user (e.g., hand 408 and/or arm gestures and/or movement), facial movement patterns (e.g., pursed lips as illustrated by representations 418a and 418b), and/or thermal image patterns (e.g., change in pixel characteristics as illustrated by representation 420a) indicative of the smoking patterns and/or characteristics, the electronic device 101 may confirm that the user is indeed smoking and initiates activation of the smoking detection mode. For example, representation 420a illustrates a thermal image of heat distribution of the user 420c and a cigarette 420b. In some examples, the electronic device 101 analyzes the heat distribution and determines a “hot spot” or non-uniformity that may indicate the presence of a cigarette 420b. In some examples, other data analyzed by the electronic device 101 to determine a smoking event includes the location of the user of the electronic device 101 and/or a time of day. For example, if the user of the electronic device 101 typically smokes outside the user's workplace building (e.g., known from user profile data and/or provided by a navigation application) and/or after a particular time of day (e.g., afternoon), the electronic device 101 may initiate activation of the smoking detection mode in response to detecting that a current time of day at the electronic device corresponds to afternoon and/or in response to detecting that the current location of the user is outside their workplace building. In some examples, the electronic device 101 may correlate the captured data of the user and/or the environment described above with confirmation that the user is smoking to further train the electronic device 101 (e.g., via machine learning) on smoking habits/characteristics of the user and detection of smoking events.

In some examples, while operating in the smoking detection mode, the electronic device monitors and logs information about the smoking event to generate insights with regards to the user's smoking habits. In some examples, the electronic device may log and/or monitor smoking data such as the frequency and/or timing of smoking sections to provide insights about the user's smoking habits (e.g., the user smokes while at work but not at home, the user does not smoke or smokes less when the outside temperature is below 40 degrees, the user smokes an average five cigarettes a day, the user spends approximately $300 a month on cigarettes, the user's heart rate increases a certain amount while smoking, and/or the like). In FIG. 4C, the electronic device 101 displays, via display 120, a user interface 422 including content related to the user's smoking event. For example, the content includes the user's smoking information of the day including a number of smoking sessions, the times of each smoking session, and the locations of the smoking sessions. In some examples, such data may be used to provide insight into the user's general wellness, or may be used as feedback to users using the smoking detection mode to pursue wellness goals. In some examples, the electronic device 101 automatically displays user interface 422 after determining that a smoking event has ended. In some examples, the electronic device 101 automatically displays user interface 422 prior to an initiation of a smoking event as determined by the electronic device 101 based on the captured data described above (e.g., thermal images, smoke pattern, user's heart rate, and/or other data described above). In some examples, the electronic device 101 displays user interface 422 in response to user input requesting to display user interface 422.

FIG. 5 illustrates a flow diagram illustrating an example process for displaying a representation of a prediction of a food being consumed by a user of the electronic device in a computer-generated environment according to some examples of the disclosure. In some examples, process 500 begins at an electronic device in communication with a display and one or more input devices. In some examples, the electronic device is optionally a head-mounted display similar or corresponding to device 201 of FIG. 2. As shown in FIG. 5, in some examples, at 502a, in accordance with a determination that one or more first criteria are satisfied, using a subset of the one or more input devices, the electronic device determines that a user of the electronic device is initiating consumption of a first object, such as consumable object 310 in FIG. 3B. In some examples, at 502b, in response to determining that the user of the electronic device is initiating consumption of the first object, the electronic device captures (502c), using the one or more input devices, audio of the consumption of the first object, such as indicated by indicators 312 and 314, respectively in FIG. 3B. In some examples, in response to determining that the user of the electronic device is initiating consumption of the first object, the electronic device obtains (502d) a first prediction of the first object based on a sound print of the first object included in the audio, such as illustrated by representation 320a in FIG. 3C, including: in accordance with a determination that the first prediction of the first object satisfies one or more second criteria, the electronic device initiates (502e) a process to analyze the one or more images of the first object, such as shown by image analyzer indicator mode 316 in FIG. 3D.

Obtaining a prediction of an object being consumed by the user of the electronic device based on a sound print avoids additional interaction between the user and the electronic device associated with manually inputting a description of the object when automatic detection and logging of the object is desired, thereby reducing errors in the interaction between the user and the electronic device and reducing inputs needed to correct such errors.

It is understood that process 500 is an example and that more, fewer, or different operations can be performed in the same or in a different order. Additionally, the operations in process 500 described above are, optionally, implemented by running one or more functional modules in an information processing apparatus such as general-purpose processors (e.g., as described with respect to FIG. 2) or application specific chips, and/or by other components of FIG. 2.

FIG. 6 is a flow diagram illustrating an example process for displaying an indication of possible non-compliance of medication in a computer-generated environment according to some examples of the disclosure. In some examples, process 600 begins at an electronic device in communication with a display and one or more input devices. In some examples, the electronic device is optionally a head-mounted display similar or corresponding to device 201 of FIG. 2. As shown in FIG. 6, in some examples, at 602a, the electronic device obtains medication information associated with a user of the electronic device, such as illustrated by user interface 342a in FIG. 3J. In some examples, at 602b, while the medication information of the user indicates a dose within a predetermined period of time, the electronic device detects, via the one or more input devices, a change in contextual information, such as the user of the electronic device interacting with user interface 344a in FIG. 3K. In some examples, in response to detecting the change in contextual information (602c), in accordance with a determination that one or more criteria are satisfied, including a criterion that is satisfied when the change in contextual information is associated with possible non-compliance of the medication, the electronic device presents (602d) an indication in a computer-generated environment of the possible non-compliance, such as representation 346 in FIG. 3L. In some examples, in response to detecting the change in contextual information, in accordance with a determination that the one or more criteria are not satisfied, the electronic device foregoes (602e) presenting the indication in the computer-generated environment, such as shown in FIG. 3M where representation 346 ceases to be displayed.

Automatically presenting an indication of possible non-compliance of medication when a change in contextual information is associated with possible non-compliance of the medication promotes the user's adherence with the prescribed regimen of the medication, thereby complying with the prescribed medicinal regimen and facilitating user actions to resolve and/or avoid such possible non-compliance.

It is understood that process 600 is an example and that more, fewer, or different operations can be performed in the same or in a different order. Additionally, the operations in process 600 described above are, optionally, implemented by running one or more functional modules in an information processing apparatus such as general-purpose processors (e.g., as described with respect to FIG. 2) or application specific chips, and/or by other components of FIG. 2.

Therefore, according to the above, some examples of the disclosure are directed to a method, comprising at an electronic device in communication with one or more displays, and one or more input devices: in accordance with a determination that one or more first criteria are satisfied, using a subset of the one or more input devices, determining a user of the electronic device is initiating consumption of a first object; and in response to determining that the user of the electronic device is initiating consumption of the first object: capturing, using the one or more input devices, audio of the consumption of the first object; and obtaining a first prediction of the first object based on a sound print of the first object included in the audio, including: in accordance with a determination that the first prediction of the first object satisfies one or more second criteria, initiating a process to analyze one or more images of the first object captured by the electronic device.

Additionally or alternatively, in some examples, the one or more first criteria include a criterion that is satisfied when a location of the electronic device, a posture of the user of the electronic device, or a time of day at the electronic device indicates initiating consumption of a first object. Additionally or alternatively, in some examples, the method further comprises: in accordance with a determination that the first prediction of the first object does not satisfy the one or more second criteria, saving the sound print of the first object to a database. Additionally or alternatively, in some examples, the method further comprises: in accordance with a determination that the first prediction of the first object does not satisfy the one or more second criteria, adding the first prediction to a digital journal accessible on the electronic device.

Additionally or alternatively, in some examples, adding the first prediction to the digital journal further includes adding contextual information associated with a physical environment surrounding the electronic device during the initiation of the consumption of the first object. Additionally or alternatively, in some examples, adding the first prediction to the digital journal further includes adding information associated with the user of the electronic device corresponding to one or more physical characteristics of the user during the initiation of the consumption of the first object. Additionally or alternatively, in some examples, obtaining the first prediction of the first object based on the sound print of the first object included in the audio includes: identifying an object from a plurality of objects that has a respective sound print that matches the sound print of the first object when a score of the object is within a predetermined score.

Additionally or alternatively, in some examples, initiating the process to analyze the one or more images of the first object includes: identifying a plurality of objects included in the one or more images; in accordance with a determination that the first prediction of the first object corresponds to a respective object of the plurality of objects, saving the sound print of the first object; and in accordance with a determination that the first prediction of the first object does not correspond to a respective object of the plurality of objects, obtaining a second prediction of the first object based on one or more of the plurality of objects included in the one or more images.

Additionally or alternatively, in some examples, obtaining the second prediction of the first object includes: identifying a second object of the plurality of objects included in the one or more images which has a respective sound print that matches the sound print of the first object when a score of the second object is within a predetermined score; and associating the second object with the sound print of the first object. Additionally or alternatively, in some examples, the method further comprises: after obtaining the first prediction of the first object, in accordance with a determination that the first prediction of the first object does not satisfy the one or more second criteria, presenting, via the one or more displays, an indication in a computer-generated environment that the first prediction has been added to a digital journal accessible on the electronic device.

Additionally or alternatively, in some examples, the method further comprises: after obtaining the first prediction of the first object, presenting, in a computer-generated environment, a request for user confirmation of the first prediction. Additionally or alternatively, in some examples, the method further comprises: after obtaining the first prediction of the first object, in accordance with a determination that the first prediction of the first object does not satisfy the one or more second criteria, presenting, via the one or more displays, a user interface of a digital journal in a computer-generated environment, wherein the user interface includes a representation of a comparison between first data corresponding to the consumption of the first object and second data corresponding to consumption of a second object, different from the first object, and wherein the representation of the comparison indicates a consumption trend.

Additionally or alternatively, in some examples, the method further comprises: after obtaining the first prediction of the first object, in accordance with a determination that the first prediction of the first object does not satisfy the one or more second criteria, presenting, via the one or more displays, a user interface of a digital journal in a computer-generated environment, wherein the user interface includes a representation of a second object, different from the first object, that is recommended for the user of the electronic device based on at least the initiation of the consumption of the first object.

Some examples of the disclosure are directed to a method, comprising at an electronic device in communication with one or more displays, and one or more input devices: obtaining medication information associated with a user of the electronic device; while the medication information of the user indicates a dose within a predetermined period of time, detecting, via the one or more input devices, a change in contextual information; and in response to detecting the change in contextual information: in accordance with a determination that one or more criteria are satisfied, including a criterion that is satisfied when the change in contextual information is associated with possible non-compliance of the medication, presenting an indication in a computer-generated environment of the possible non-compliance; and in accordance with a determination that the one or more criteria are not satisfied, foregoing presenting the indication in the computer-generated environment.

Additionally or alternatively, in some examples, the contextual information includes a detected activity of the user of the electronic device during the predetermined period of time of the dose. Additionally or alternatively, in some examples, the contextual information includes information associated with the user of the electronic device corresponding to one or more physical characteristics of the user during the predetermined period of time of the dose. Additionally or alternatively, in some examples, the contextual information includes location information corresponding to a physical environment of the user of the electronic device during the predetermined period of time of the dose. Additionally or alternatively, in some examples, the indication includes information indicative of an end of the predetermined period of time of the dose.

Additionally or alternatively, in some examples, the indication includes information corresponding to a recommendation based on at least the predetermined period of time of the dose. Additionally or alternatively, in some examples, the method further comprises: while presenting the indication in the computer-generated environment of the possible non-compliance, detecting, via the one or more input devices, a second change in contextual information; and in response to detecting the second change in contextual information: in accordance with a determination that one or more second criteria are satisfied, including a criterion that is satisfied when the second change in contextual information is associated with possible non-compliance of the medication, maintaining presentation of the indication in the computer-generated environment of the possible non-compliance; and in accordance with a determination that the one or more second criteria are not satisfied, ceasing presentation of the indication in the computer-generated environment.

Additionally or alternatively, in some examples, the method further comprises: in response to detecting the change in contextual information: in accordance with a determination that one or more second criteria are satisfied, including a second criterion that is satisfied when a current time of day at the electronic device is within the predetermined period of time of the dose, presenting a second indication in the computer-generated environment prompting the user of the electronic device to initiate consumption of the medication; and in accordance with a determination that the one or more second criteria are not satisfied, foregoing presenting the second indication.

Additionally or alternatively, in some examples, the method further comprises: in response to detecting the change in contextual information: in accordance with a determination that one or more second criteria are satisfied, including a criterion that is satisfied when an opportunity to consume the medication is detected, presenting a second indication in the computer-generated environment prompting the user of the electronic device to initiate consumption of the medication; and in accordance with a determination that the one or more second criteria are not satisfied, foregoing presenting the second indication.

Some examples of the disclosure are directed to an electronic device, comprising: one or more processors; memory; and one or more programs stored in the memory and configured to be executed by the one or more processors, the one or more programs including instructions for performing any of the above methods.

Some examples of the disclosure are directed to a non-transitory computer readable storage medium storing one or more programs, the one or more programs comprising instructions, which when executed by one or more processors of an electronic device, cause the electronic device to perform any of the above methods.

Some examples of the disclosure are directed to an electronic device, comprising one or more processors, memory, and means for performing any of the above methods.

Some examples of the disclosure are directed to an information processing apparatus for use in an electronic device, the information processing apparatus comprising means for performing any of the above methods.

The present disclosure contemplates that in some instances, the data utilized may include personal information data that uniquely identifies or can be used to contact or locate a specific person. Such personal information data can include demographic data, content consumption activity, location-based data, telephone numbers, email addresses, twitter ID's, home addresses, data or records relating to a user's health or level of fitness (e.g., vital signs measurements, medication information, exercise information), date of birth, or any other identifying or personal information. Specifically, as described herein, one aspect of the present disclosure is tracking a user's biometric data.

The present disclosure recognizes that the use of such personal information data, in the present technology, can be used to the benefit of users. For example, personal information data may be used to display a visual indication based on changes in a user's biometric data. For example, the visual indication includes a recommendation for the user to visit or contact a health professional as a result of the detecting an abnormality compared with baseline biometric data.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates examples in which users selectively block the use of, or access to, personal information data. That is, the present disclosure contemplates that hardware and/or software elements can be provided to prevent or block access to such personal information data. For example, the present technology can be configured to allow users to select to “opt in” or “opt out” of participation in the collection of personal information data during registration for services or anytime thereafter. In another example, users can select not to enable recording of personal information data in a specific application (e.g., first application and/or second application). In addition to providing “opt in” and “opt out” options, the present disclosure contemplates providing notifications relating to the access or use of personal information. For instance, a user may be notified upon initiating collection that their personal information data will be accessed and then reminded again just before personal information data is accessed by the device(s).

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

The foregoing description, for purpose of explanation, has been described with reference to specific examples. However, the illustrative discussions above are not intended to be exhaustive or to limit the disclosure to the precise forms disclosed. Many modifications and variations are possible in view of the above teachings. The examples were chosen and described in order to best explain the principles of the disclosure and its practical applications, to thereby enable others skilled in the art to best use the disclosure and various described examples with various modifications as are suited to the particular use contemplated.

What is claimed is:

1. A method comprising:
at an electronic device in communication with one or more displays and one or more input devices:
in accordance with a determination that one or more first criteria are satisfied, using a subset of the one or more input devices, determining a user of the electronic device is initiating consumption of a first object; and
in response to determining that the user of the electronic device is initiating consumption of the first object:
capturing, using the one or more input devices, audio of the consumption of the first object; and
obtaining a first prediction of the first object based on a sound print of the first object included in the audio, including:
in accordance with a determination that the first prediction of the first object satisfies one or more second criteria, initiating a process to analyze one or more images of the first object captured by the electronic device.

2. The method of claim 1, wherein the one or more first criteria include a criterion that is satisfied when a location of the electronic device, a posture of the user of the electronic device, or a time of day at the electronic device indicates initiating consumption of a first object.

3. The method of claim 1, further comprising:
in accordance with a determination that the first prediction of the first object does not satisfy the one or more second criteria, saving the sound print of the first object to a database.

4. The method of claim 1, further comprising:
in accordance with a determination that the first prediction of the first object does not satisfy the one or more second criteria, adding the first prediction to a digital journal accessible on the electronic device.

5. The method of claim 4, wherein adding the first prediction to the digital journal further includes adding contextual information associated with a physical environment surrounding the electronic device during the initiation of the consumption of the first object.

6. The method of claim 4, wherein adding the first prediction to the digital journal further includes adding information associated with the user of the electronic device corresponding to one or more physical characteristics of the user during the initiation of the consumption of the first object.

7. The method of claim 1, wherein obtaining the first prediction of the first object based on the sound print of the first object included in the audio includes:
identifying an object from a plurality of objects that has a respective sound print that matches the sound print of the first object when a score of the object is within a predetermined score.

8. The method of claim 1, wherein initiating the process to analyze the one or more images of the first object includes:
identifying a plurality of objects included in the one or more images;
in accordance with a determination that the first prediction of the first object corresponds to a respective object of the plurality of objects, saving the sound print of the first object; and
in accordance with a determination that the first prediction of the first object does not correspond to a respective object of the plurality of objects, obtaining a second prediction of the first object based on one or more of the plurality of objects included in the one or more images.

9. The method of claim 8, wherein obtaining the second prediction of the first object includes:
identifying a second object of the plurality of objects included in the one or more images which has a respective sound print that matches the sound print of the first object when a score of the second object is within a predetermined score; and
associating the second object with the sound print of the first object.

10. The method of claim 1, further comprising:
after obtaining the first prediction of the first object, in accordance with a determination that the first prediction of the first object does not satisfy the one or more second criteria, presenting, via the one or more displays, an indication in a computer-generated environment that the first prediction has been added to a digital journal accessible on the electronic device.

11. The method of claim 1, further comprising:
after obtaining the first prediction of the first object, presenting, in a computer-generated environment, a request for user confirmation of the first prediction.

12. The method of claim 1, further comprising:
after obtaining the first prediction of the first object, in accordance with a determination that the first prediction of the first object does not satisfy the one or more second criteria, presenting, via the one or more displays, a user interface of a digital journal in a computer-generated environment, wherein the user interface includes a representation of a comparison between first data corresponding to the consumption of the first object and second data corresponding to consumption of a second object, different from the first object, and wherein the representation of the comparison indicates a consumption trend.

13. The method of claim 1, further comprising:
after obtaining the first prediction of the first object, in accordance with a determination that the first prediction of the first object does not satisfy the one or more second criteria, presenting, via the one or more displays, a user interface of a digital journal in a computer-generated environment, wherein the user interface includes a representation of a second object, different from the first object, that is recommended for the user of the electronic device based on at least the initiation of the consumption of the first object.

14. An electronic device comprising:
one or more processors;
memory; and
one or more programs stored in the memory and configured to be executed by the one or more processors, the one or more programs including instructions for:
in accordance with a determination that one or more first criteria are satisfied, using a subset of one or more input devices, determining a user of the electronic device is initiating consumption of a first object; and
in response to determining that the user of the electronic device is initiating consumption of the first object:
capturing, using the one or more input devices, audio of the consumption of the first object; and
obtaining a first prediction of the first object based on a sound print of the first object included in the audio, including:
in accordance with a determination that the first prediction of the first object satisfies one or more second criteria, initiating a process to analyze one or more images of the first object captured by the electronic device.

15. The electronic device of claim 14, wherein the one or more first criteria include a criterion that is satisfied when a location of the electronic device, a posture of the user of the electronic device, or a time of day at the electronic device indicates initiating consumption of a first object.

16. The electronic device of claim 14, wherein the one or more programs including instructions for:
in accordance with a determination that the first prediction of the first object does not satisfy the one or more second criteria, saving the sound print of the first object to a database.

17. The electronic device of claim 14, wherein the one or more programs including instructions for:
in accordance with a determination that the first prediction of the first object does not satisfy the one or more second criteria, adding the first prediction to a digital journal accessible on the electronic device.

18. A non-transitory computer readable storage medium storing one or more programs, the one or more programs comprising instructions, which when executed by one or more processors of an electronic device, cause the electronic device to perform a method comprising:
in accordance with a determination that one or more first criteria are satisfied, using a subset of one or more input devices, determining a user of the electronic device is initiating consumption of a first object; and
in response to determining that the user of the electronic device is initiating consumption of the first object:
capturing, using the one or more input devices, audio of the consumption of the first object; and
obtaining a first prediction of the first object based on a sound print of the first object included in the audio, including:
in accordance with a determination that the first prediction of the first object satisfies one or more second criteria, initiating a process to analyze one or more images of the first object captured by the electronic device.

19. The non-transitory computer readable storage medium of claim 18, wherein the one or more first criteria include a criterion that is satisfied when a location of the electronic device, a posture of the user of the electronic device, or a time of day at the electronic device indicates initiating consumption of a first object.

20. The non-transitory computer readable storage medium of claim 18, wherein the one or more programs comprise instructions for:
in accordance with a determination that the first prediction of the first object does not satisfy the one or more second criteria, saving the sound print of the first object to a database.
